(19) United States
(12) Patent Application Publication
(10) Pub. No.: US 2021/0403993 A1
(43) Pub. Date: Dec. 30, 2021

(54) CATALYTICALLY CONTROLLED SEQUENCING BY SYNTHESIS TO PRODUCE SCARLESS DNA

(71) Applicant: ILLUMINA, INC., San Diego, CA (US)

(72) Inventors: Kaitlin PUGLIESE, San Diego, CA (US); Sergio PEISAJOVICH, San Diego, CA (US); Jeffrey MANDELL, San Diego, CA (US); Seth MCDONALD, San Diego, CA (US)

(21) Appl. No.: 17/361,988

(22) Filed: Jun. 29, 2021

Related U.S. Application Data
(60) Provisional application No. 63/045,914, filed on Jun. 30, 2020.

(51) Int. Cl. C12Q 1/6869

(52) U.S. Cl. CPC C12Q 1/6869

(57) ABSTRACT

The present disclosure relates to methods comprising (a) contacting a polymerase with a template polynucleotide and a plurality of free nucleotides, wherein the template polynucleotide is hybridized to a complementary polynucleotide comprising a 3′ end overhung by a 5′ terminal fragment of the template polynucleotide, and the plurality of free nucleotides comprise a compound Formula (I); wherein said contacting occurs under a complexation condition, the complexation condition effective to form a complex but not effective to form polymerization, wherein the complex comprises the polymerase, the template polynucleotide, the complementary polynucleotide, and one of the plurality of free nucleotides that is complementary to a first nucleotide of the 5′ terminal fragment of the template polynucleotide; (b) detecting a signal from the fluorescent label; and (c) exposing the complex to a polymerization condition.

### CROSS REFERENCE TO RELATED APPLICATION

This application claims benefit of U.S. Provisional Patent Application Ser. No. 63/045,914, filed Jun. 30, 2020, which is hereby incorporated by reference in its entirety.

### FIELD

The present disclosure relates generally to methods for catalytically controlled sequencing by synthesis to produce scarless DNA.

### BACKGROUND

Many current sequencing platforms use “sequencing by synthesis” (“SBS”) technology and fluorescence based methods for detection. Alternative sequencing methods that allow for more cost effective, rapid, and convenient sequencing and nucleic acid detection are desirable as complements to SBS.

Current SBS technology uses nucleotides that are modified at two positions: 1) the 3′ hydroxyl (3′-OH) of deoxyribose, and 2) the 5-position of pyrimidines or 7-position of purines of nitrogenous bases (A, T, C, G). The 3′-OH group is blocked with an azidomethyl group to create reversible nucleotide terminators. This may prevent further elongation after the addition of a single nucleotide. Each of the nitrogenous bases is separately modified with a fluorophore to provide a fluorescence readout which identifies the single base incorporation. Subsequently, the 3′-OH blocking group and the fluorophore are removed and the cycle repeats.

The current cost of the modified nucleotides may be high due to the synthetic challenges of modifying both the 3′-OH of deoxyribose and the nitrogenous base. There are several possible methods to reduce the cost of the modified nucleotides. One method is to move the readout label to the 5′-terminal phosphate instead of the nitrogenous base. In one example, this removes the need for a separate cleavage step, and allows for real time detection of the incoming nucleotide. During incorporation, the pyrophosphate together with the tag is released as a by-product of the elongation process, thus a cleavable linkage is not involved.

Current fully functionalized nucleotide (“ffNs”) used in SBS carry a dye label on the nucleobase, which may be cleaved in a separate step during each cycle. In some instances, such cleavage may chemically modify the nucleotide at or near where the dye label was attached, leaving behind a “scar” on the DNA, in some instances perhaps disadvantageously affecting binding of the produced DNA to the SBS polymerase, downstream sequencing metrics, or other aspects of an SBS process.

The present disclosure is directed to overcoming these and other deficiencies in the art.

### SUMMARY

A first aspect relates to a method. The method includes (a) contacting a polymerase with a template polynucleotide and a plurality of free nucleotides, wherein the template polynucleotide is hybridized to a complementary polynucleotide including a 3′ end overhung by a 5′ terminal fragment of the template polynucleotide, and the plurality of free nucleotides include a compound of Formula (I):

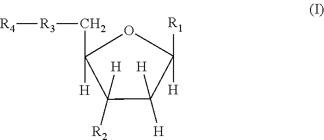

wherein R₁ includes a nitrogenous base selected from adenine, guanine, cytosine, thymine and uracil; R₂ includes —O—R₂ wherein R₂ is H or Z where Z is a removable protecting group comprising an azido group; R₃ includes a linker including three or more phosphate groups; and R₄ includes a fluorescent label; wherein said contacting occurs under a complexation condition, the complexation condition effective to form a complex but not effective to form polymerization, wherein the complex includes the polymerase, the template polynucleotide, the complementary polynucleotide, and one of the plurality of free nucleotides that is complementary to a first nucleotide of the 5′ terminal fragment of the template polynucleotide; (b) detecting a signal from the fluorescent label; and (c) exposing the complex to a polymerization condition.

In one embodiment, R₂ consists of —O—R₂ wherein R₂ is H or Z wherein Z is a removable protecting group comprising an azido group. In another embodiment, the template polynucleotide is one of a plurality of template polynucleotides attached to a substrate. In one embodiment, the plurality of template polynucleotides attached to the substrate include a cluster of copies of a library polynucleotide. In another embodiment, the method further includes repeating steps a) through c) one or more times.

In one embodiment, the polymerization condition includes a concentration of Mg²⁺ ions, wherein the concentration of Mg²⁺ ions is in a range of about 0.1 mM to about 10 mM, or a concentration of Mn²⁺ ions, wherein the concentration of Mn²⁺ ions is in a range of about 0.1 mM to about 10 mM. In another embodiment, the complexation condition includes a non-catalytic metal cation. In one embodiment, the non-catalytic metal cation is selected from the group consisting of one or more of Ca²⁺, Zn²⁺, Co²⁺, Ni²⁺, Eu²⁺, Sr²⁺, Ba²⁺, Fe²⁺, and Eu²⁺. In yet another embodiment, the concentration of the non-catalytic metal cation is less than or equal to about 10 mM.

In one embodiment, the complexation condition includes a chelating agent. In one embodiment, the chelating agent is selected from the group consisting of ethylene glycol-bis(β-aminoethyl ether)-N,N,N′,N′-tetraacetic acid (EGTA), nitriloacetic acid, tetrasodium iminodisuccinate, ethylene glycol tetraacetic acid, polyaspartic acid, ethylenediamine-N,N′-disuccinic acid (EDDS), methylglycindiacetic acid (MGDA), and a combination thereof.

In one embodiment, the complexation condition further includes an inhibitor selected from the group consisting of a non-competitive inhibitor, a competitive inhibitor, and a combination thereof. In another embodiment, the complexation condition includes a pH that is less than about 6.

In another embodiment, the polymerization condition includes a pH that is greater than or equal to about 6. In one embodiment, the complexation condition includes a non-competitive inhibitor. In one embodiment, the non-competitive inhibitor is selected from the group consisting of an aminoglycoside, a pyrophosphate analog, a melanin, a phosphonoacetate, a hypophosphate, a rifamycin, and a combination thereof.

In one embodiment, the complexation condition includes a competitive inhibitor. In one embodiment, the competitive inhibitor is selected from the group consisting of aphidicolin, beta-D-arabinofuranosyl-CTP, amiloride, dehydroaltenusin, and a combination thereof. In one embodiment, the complexation condition includes a solvent additive. In one embodiment, the solvent additive is selected from the group consisting of ethanol, methanol, tetrahydrofuran, dioxane, dimethylamine, dimethylformamide, dimethyl sulfoxide, lithium, L-cysteine, and a combination thereof. In another embodiment, the complexation condition includes deuterium.

In one embodiment, the 3′-hydroxy blocking group includes a reversible terminator. In another embodiment, the reversible terminator includes an azidomethyl group or an acetal group. In yet another embodiment, the method further includes removing the reversible terminator after the 3′ end of the complementary polynucleotide is covalently bonded to a phosphate group of the linker. In yet another embodiment, the free nucleotide further includes a non-bridging thiol or a bridging nitrogen. In one embodiment, the polymerase includes a mutation. In another embodiment, the mutation modifies speed of one or more of steps a) through c).

Current ffNs used in SBS carry a dye label on the nucleobase, which must be cleaved in a separate step during each cycle. This cleavage leaves behind a “scar” on the DNA, potentially affecting binding of the produced DNA to the SBS polymerase and downstream sequencing metrics. By moving the fluorescence tag (or any other detection tag) away from the nucleobase to the 5′ terminal phosphate and carefully controlling enzyme catalysis, incorporation of the nucleotide will result in the release of the detection tag completely, leaving behind scarless DNA, that is DNA without deleterious modifications of its nucleobase that would otherwise resulted from removal of a dye label therefrom.

### BRIEF DESCRIPTION OF THE DRAWINGS

FIGS. 1A-1F depict a schematic representation of a scarless SBS cycle. FIG. 1A shows that the polymerase is bound to primed DNA that is clustered on a flow cell surface. In FIG. 1B, the nucleotide substrate carrying a 5′-phosphate label is introduced under conditions which control catalysis, pausing polymerase incorporation kinetics and retaining the label on the 5′ phosphate. Depending on the mode of detection, excess substrates may be washed away after binding. The nucleotide may optionally carry a 3′-block to prevent multiple nucleotide incorporation events upon introduction of catalytic conditions. In FIG. 1C, the signal per cluster is measured while the nucleotide substrate and its 5′-phosphate label are still bound, prior to catalysis. FIG. 1D shows that the conditions of the flow cell are changed such that catalysis can be promoted and the 5′ phosphate label is released from the cluster. Presence of a 3′-block in embodiments that do not employ washing away of excess substrate after nucleotide binding will be necessary here to enable only single extension events. In FIG. 1E, the resulting DNA product contains a natural nucleotide. FIG. 1F shows that in some embodiments, which employ a nucleotide substrate with a 3′-block, a subsequent deblocking step may be needed to prepare the cluster for subsequent cycles.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits and advantages described herein.

### DETAILED DESCRIPTION

A first aspect relates to a method. The method includes (a) contacting a polymerase with a template polynucleotide and a plurality of free nucleotides, wherein the template polynucleotide is hybridized to a complementary polynucleotide including a 3′ end overhung by a 5′ terminal fragment of the template polynucleotide, and the plurality of free nucleotides include a compound of Formula (I):

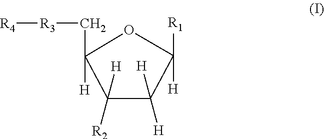

wherein R₁ includes a nitrogenous base selected from adenine, guanine, cytosine, thymine and uracil; R₂ includes —O—R₂ where R₂ is H or Z wherein Z is a removable protecting group comprising an azido group; R₃ includes a linker including three or more phosphate groups; and R₄ includes a fluorescent label; wherein said contacting occurs under a complexation condition, the complexation condition effective to form a complex but not effective to form polymerization, wherein the complex includes the polymerase, the template polynucleotide, the complementary polynucleotide, and one of the plurality of free nucleotides that is complementary to a first nucleotide of the 5′ terminal fragment of the template polynucleotide; (b) detecting a signal from the fluorescent label; and (c) exposing the complex to a polymerization condition.

It is to be appreciated that certain aspects, modes, embodiments, variations, and features of the present disclosure are described below in various levels of detail in order to provide a substantial understanding of the present technology. Unless otherwise noted, all technical and scientific terms used herein generally have the same meaning as commonly understood by one of ordinary skill in the art. The use of the term “including” as well as other forms is not limiting. The use of the term “having” as well as other forms is not limiting. As used in this disclosure, whether in a transitional phrase or in the body of the claim, the terms “comprise(s)” and “comprising” are to be interpreted as having an open-ended meaning. That is, the terms are to be interpreted synonymously with the phrases “having at least” or “including at least.”

The terms “substantially”, “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, fluctuations can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable sub-combination.

The terms “connect”, “contact”, and/or “coupled” include a variety of arrangements and assemblies. These arrangements and techniques include, but are not limited to, (1) the direct joining of one component and another component with no intervening components therebetween (i.e., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (optionally with the presence of one or more additional components therebetween). Components that are in direct physical contact with one another may or may not be in electrical contact and/or fluid contact with one another. Moreover, two components that are electrically connected, electrically coupled, optically connected, optically coupled, fluidly connected, or fluidly coupled may or may not be in direct physical contact, and one or more other components may be positioned between those two connected components.

As described herein, the term “array” may include a population of conductive channels or molecules that may attach to one or more solid-phase substrates such that the conductive channels or molecules can be differentiated from one another based on their location. An array as described herein may include different molecules that are each located at a different identifiable location (e.g., at different conductive channels) on a solid-phase substrate. Alternatively, an array may include separate solid-phase substrates each bearing a different molecule, where the different probe molecules can be identified according to the locations of the solid-phase substrates on a surface to which the solid-phase substrates attach or based on the locations of the solid-phase substrates in a liquid such as a fluid stream. Examples of arrays where separate substrates are located on a surface include wells having beads as described in U.S. Pat. No. 6,355,431, U.S. Pat. Publ. No. 2002/0102578, and WO 00/63437, all of which are hereby incorporated by reference in their entirety. Molecules of the array can be nucleic acid primers, nucleic acid probes, nucleic acid templates, or nucleic acid enzymes such as polymerases and exonucleases.

As described herein, the term “attached” may include when two things are joined, fastened, adhered, connected, or bound to one another. A reaction component, like a polymerase, can be attached to a solid phase component, like a conductive channel, by a covalent or a non-covalent bond. As described herein, the phrase “covalently attached” or “covalently bonded” refers to forming one or more chemical bonds that are characterized by the sharing of pairs of electrons between atoms. A non-covalent bond is one that does not involve the sharing of pairs of electrons and may include, for example, hydrogen bonds, ionic bonds, van der Waals forces, hydrophilic interactions, and hydrophobic interactions.

As used herein, any “R” group(s) represents substituents that may be attached to an indicated atom. An R group may be substituted or unsubstituted. If two R groups are described as “together with the atoms to which they are attached” forming a ring or ring system, it means that the collective unit of the atoms, intervening bonds and the two R groups are the recited ring.

C₁ to C₂₀ hydrocarbon includes alkyl, cycloalkyl, polycycloalkyl, alkenyl, alkynyl, aryl, and combinations thereof. Examples include benzyl, phenethyl, propargyl, allyl, cyclohexylmethyl, adamantyl, camphoryl, and naphthylethyl. Hydrocarbon refers to any substituent included of hydrogen and carbon as the only elemental constituents.

The term “alkyl” includes an aliphatic hydrocarbon group which may be straight or branched having about 1 to about 23 carbon atoms in the chain. For example, straight or branched carbon chain could have 1 to 10 carbon atoms or 1 to 6 carbon atoms. Branched means that one or more lower alkyl groups such as methyl, ethyl or propyl are attached to a linear alkyl chain. Alkyl includes a hydrocarbon that is fully saturated (i.e., contains no double or triple bonds) and combinations thereof. (e.g.,1 to 10 carbon atoms, such as 1 to 6 carbon atoms). Examples of alkyl groups include but are not limited to methyl, ethyl, propyl, n-propyl, isopropyl, butyl, isobutyl, n-butyl, s-butyl, t-butyl, n-pentyl, and 3-pentyl. An alkyl group may have between 1 to about 23 carbon atoms (whenever it appears herein, a numerical range such as “1 to 23” refers to each integer in the given range; e.g., “1 to 23 carbon atoms” means that the alkyl group may consist of 1 carbon atom, 2 carbon atoms, 3 carbon atoms, 4 carbon atoms, 5 carbon atoms, etc., and up to and including 23 carbon atoms, although the present disclosure also covers the occurrence of the term “alkyl” where no numerical range is designated). For example, “C₁-C₆ alkyl” indicates that there are between one and six carbon atoms in the alkyl chain (i.e., the alkyl chain is selected from the group consisting of methyl, ethyl, propyl, iso-propyl, n-butyl, iso-butyl, sec-butyl, and t-butyl).

As described herein, “alkenyl” refers to a straight or branched hydrocarbon chain containing one or more double bonds. An alkenyl group may have about 2 to about 23 carbon atoms, although the present description also covers the occurrence of the term “alkenyl” where no numerical range is designated. The alkenyl group may also be a medium size alkenyl having 2 to 9 carbon atoms. The alkenyl group could also be a lower alkenyl having between 2 and 6 carbon atoms. For example, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups may include, but are not limited to, ethenyl, propenyl, butenyl, pentenyl, and hexenyl.

As described herein, “alkynyl” includes a straight or branched hydrocarbon chain containing one or more triple bonds. An alkynyl group may have between about 2 and about 23 carbon atoms, although the present description also includes the occurrence of the term “alkynyl” where no numerical range is designated. As an example, “C₂-C₆ alkynyl” indicates that may be between two and six carbon atoms in the alkynyl chain (i.e., the alkynyl chain may be selected from the group consisting of ethynyl, propyn-1-yl, propyn-2-yl, butyn-1-yl, butyn-3-yl, butyn-4-yl, and 2-butynyl). Typical alkynyl groups may include, but are not limited to, ethynyl, propynyl, butynyl, pentynyl, and hexynyl, and the like.

As described herein, “heteroalkyl” may include a straight or branched hydrocarbon chain containing one or more heteroatoms, that is, an element other than carbon, including but not limited to, nitrogen, oxygen, and sulfur, in the chain backbone. A heteroalkyl group may have between 1 and 20 carbon atoms, although the present disclosure also includes the occurrence of the term “heteroalkyl” where no numerical range is designated. For example, “C₄-C₆ heteroalkyl” may indicate that there are between four and six carbon atoms in the heteroalkyl chain and additionally one or more heteroatoms in the backbone of the chain.

Aromatic as described herein refers to a ring or ring system having a conjugated pi electron system and includes both carbocyclic aromatic (e.g., phenyl) and heterocyclic aromatic groups (e.g., pyridine). Aromatics may include monocyclic or fused-ring polycyclic (i.e., rings which share adjacent pairs of atoms) groups provided the entire ring system is aromatic.

“Aryl” as described herein includes an aromatic ring or ring system (e.g., two or more fused rings that share two adjacent carbon atoms) containing only carbon in the ring backbone. The present disclosure also includes the occurrence of the term “aryl” where no numerical range is designated. In one embodiment, the aryl group has between 6 and 10 carbon atoms. An aryl group may be designated as “C₆-C₁₀ aryl” for example. Representative aryl groups include, but are not limited to, phenyl, naphthyl, azulenyl, and anthracenyl.

An “aralkyl” or “arylalkyl” as described herein may include an aryl group connected, as a substituent, via an alkylene group, such as for example C₇-C₁₄ aralkyl and the like, including but not limited to benzyl, 2-phenylethyl, 3-phenylpropyl, and naphthylalkyl.

The term “heteroaryl” includes an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen, or sulfur. In the case of multicyclic ring system, only one of the rings needs to be aromatic for the ring system to be defined as “heteroaryl.” The heteroaryl group may have between 5-18 ring members (i.e., the number of atoms making up the ring backbone, including carbon atoms and heteroatoms), although the present disclosure also includes the occurrence of the term “heteroaryl” where no numerical range is designated. Preferred heteroaryls contain between about 5 to 10 ring atoms, or between about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include thienyl, phthalazinyl, pyridinyl, benzoxazolyl, benzothienyl, pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-15 a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

A “heteroaralkyl” or “heteroarylalkyl” refers to a heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl.

Unless otherwise specified, the term “carbocycle” is intended to include ring systems in which the ring atoms are all carbon but of any oxidation state. When the carbocyclyl is a ring system, two or more rings may be joined together in a fused, bridged, or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, and the present use of the term “carbocyclyl” also includes when no numerical range is designated. Thus (C₃-C₁₂) carbocycle, for example, refers to both non-aromatic and aromatic systems, including such systems as cyclopropane, benzene, and cyclohexene. Carbocycle, if not otherwise limited, refers to monocycles, bicycles, and polycycles.

As used herein, “cycloalkyl” means a fully saturated carbocyclyl ring or ring system. Cycloalkyl is a subset of hydrocarbon and includes cyclic hydrocarbon groups of from 3 to 8 carbon atoms. Examples of cycloalkyl groups include c-propyl, c-butyl, c-pentyl, and norbornyl (e.g., cyclopropyl, cyclobutyl, cyclopentyl, and cyclohexyl).

As used herein, the term “C₁-C₆” includes C₁, C₂, C₃, C₄, C₅, and C₆, and a range defined by any of the two numbers. For example, C₁-C₆ alkyl includes C₁, C₂, C₃, C₄, C₅, and C₆ alkyl, C₂-C₆ alkyl, C₁-C₃ alkyl, etc. Similarly, C₂-C₆ alkenyl includes C₁, C₂, C₃, C₄, C₅, and C₆ alkenyl, C₂-C₅ alkenyl, C₃-C₄ alkenyl, etc.; and C₂-C₆ alkynyl includes C₂, C₃, C₄, C₅, and C₆ alkynyl, C₂-C₅ alkynyl, C₃-C₄ alkynyl, etc. C₃-C₅ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “heterocyclyl” or “heterocycle” refers to a stable 3- to 18-membered ring (radical) which consists of carbon atoms and from one to five heteroatoms selected from the group consisting of nitrogen, oxygen and sulfur. For purposes of this disclosure, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the occurrence of the term “heterocyclyl” where no numerical range is designated is included. Examples of such heterocycles include, without limitation, acridinyl, carbazolyl, imidazolinyl, oxepanyl, thiepanyl, dioxopiperazinyl, pyrrolidonyl, pyrrolidionyl, oxiranyl, azepinyl, azocanyl, pyranyl dioxolanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, thiamorpholinyl sulfone, and tetrahydroquinoline. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

The term “halogen” or “halo” as used herein, may include any one of the radio-stable atoms of column 7 of the Periodic Table of the Elements, e.g., fluorine, chlorine, bromine, or iodine.

The term “substituted” or “substitution” of an atom means that one or more hydrogen on the designated atom is replaced with a selection from the indicated group, provided that the designated atom's normal valency is not exceeded. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents. Wherever a group is described as “optionally substituted” that group may be substituted with the above substituents.

“Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture.

The term “optionally substituted” is used to indicate that a group may have substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. “Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture.

The term “hydroxy” as used herein includes a —OH group.

As described herein, the terms “polynucleotide” or “nucleic acids” refer to deoxyribonucleic acid (DNA), ribonucleic acid (RNA), or analogs of either DNA or RNA made from nucleotide analogs. The terms as used herein also encompasses cDNA, that is complementary, or copy DNA produced from an RNA template, for example by the action of reverse transcriptase. In one embodiment, the nucleic acid to be analyzed, for example by sequencing through use of the described systems, is immobilized on a substrate (e.g., a substrate within a flow cell or one or more beads upon a substrate such as a flow cell, etc.). The term immobilized as used herein is intended to encompass direct or indirect, covalent, or non-covalent attachment, unless indicated otherwise, either explicitly or by context. The analytes (e.g., nucleic acids) may remain immobilized or attached to the support under conditions in which it is intended to use the support, such as in applications requiring nucleic acid sequencing. In one embodiment, the template polynucleotide is one of a plurality of template polynucleotides attached to a substrate. In one embodiment, the plurality of template polynucleotides attached to the substrate include a cluster of copies of a library polynucleotide as described herein.

Nucleic acids include naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art such as peptide nucleic acid (PNA) or locked nucleic acid (LNA). Naturally occurring nucleic acids generally have a deoxyribose sugar (e.g. found in deoxyribonucleic acid (DNA)) or a ribose sugar (e.g. found in ribonucleic acid (RNA)).

In RNA, the sugar is a ribose, and in DNA a deoxyribose, i.e., a sugar lacking a hydroxyl group that is present in ribose. The nitrogen containing heterocyclic base can be purine or pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose may be bonded to N-1 of a pyrimidine or N-9 of a purine.

A nucleic acid can contain any of a variety of analogs of these sugar moieties that are known in the art. A nucleic acid can include native or non-native bases. A native deoxyribonucleic acid can have one or more bases selected from the group consisting of adenine, thymine, cytosine, or guanine and a ribonucleic acid can have one or more bases selected from the group consisting of uracil, adenine, cytosine or guanine. Useful non-native bases that can be included in a nucleic acid are known in the art. In the present disclosure, R₁ includes a nitrogenous base selected from adenine, guanine, cytosine, thymine, and uracil.

The term nucleotide as described herein may include natural nucleotides, analogs thereof, ribonucleotides, deoxyribonucleotides, dideoxyribonucleotides and other molecules known as nucleotides. As described herein, a nucleotide may include a nitrogen containing heterocyclic base, a sugar, and one or more phosphate groups. Nucleotides may be monomeric units of a nucleic acid sequence, for example to identify a subunit present in a DNA or RNA strand. A nucleotide may also include a molecule that is not necessarily present in a polymer, for example, a molecule that is capable of being incorporated into a polynucleotide in a template dependent manner by a polymerase. A nucleotide may include a nucleoside unit having, for example, 0, 1, 2, 3 or more phosphates on the 5′ carbon. Tetraphosphate nucleotides, pentaphosphate nucleotides, and hexaphosphate nucleotides may be useful, as may be nucleotides with more than 6 phosphates, such as 7, 8, 9, 10, or more phosphates, on the 5′ carbon. Examples of naturally occurring nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP.

Non-natural nucleotides include nucleotide analogs, such as those that are not present in a natural biological system or not substantially incorporated into polynucleotides by a polymerase in its natural milieu, for example, in a non-recombinant cell that expresses the polymerase. Non-natural nucleotides include those that are incorporated into a polynucleotide strand by a polymerase at a rate that is substantially faster or slower than the rate at which another nucleotide, such as a natural nucleotide that base-pairs with the same Watson-Crick complementary base, is incorporated into the strand by the polymerase. For example, a non-natural nucleotide may be incorporated at a rate that is at least 2 fold different, 5 fold different, 10 fold different, 25 fold different, 50 fold different, 100 fold different, 1000 fold different, 10000 fold different, or more when compared to the incorporation rate of a natural nucleotide. A non-natural nucleotide can be capable of being further extended after being incorporated into a polynucleotide. Examples include, nucleotide analogs having a 3′ hydroxyl or nucleotide analogs having a reversible terminator moiety at the 3′ position that can be removed to allow further extension of a polynucleotide that has incorporated the nucleotide analog. Examples of reversible terminator moieties are described, for example, in U.S. Pat. Nos. 7,427,673, 7,414,116, and 7,057,026, as well as WO 91/06678 and WO 07/123744, each of which is hereby incorporated by reference in its entirety. It will be understood that in some examples a nucleotide analog having a 3′ terminator moiety or lacking a 3′ hydroxyl (such as a dideoxynucleotide analog) can be used under conditions where the polynucleotide that has incorporated the nucleotide analog is not further extended. In some examples, nucleotide(s) may not include a reversible terminator moiety, or the nucleotides(s) will not include a non-reversible terminator moiety or the nucleotide(s) will not include any terminator moiety at all.

As used herein, a “nucleoside” is structurally similar to a nucleotide, but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside including a ribose moiety and a deoxyribonucleoside including a deoxyribose moiety. A modified pentose moiety is a pentose moiety in which an oxygen atom has been replaced with a carbon and/or a carbon has been replaced with a sulfur or an oxygen atom. A “nucleoside” is a monomer that may have a substituted base and/or sugar moiety.

The term “purine base” is used herein in its ordinary sense as understood by those skilled in the art, and includes its tautomers. Similarly, the term “pyrimidine base” is used herein in its ordinary sense as understood by those skilled in the art, and includes its tautomers. A non-limiting list of optionally substituted purine-bases includes purine, adenine, guanine, hypoxanthine, xanthine, alloxanthine, 7-alkylguanine (e.g. 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

The term substrate (or solid support), as described herein, may include any inert substrate or matrix to which nucleic acids can be attached, such as for example glass surfaces, plastic surfaces, latex, dextran, polystyrene surfaces, polypropylene surfaces, polyacrylamide gels, gold surfaces, and silicon wafers. For example, a substrate may be a glass surface (e.g., a planar surface of a flow cell channel). In one embodiment, a substrate may include an inert substrate or matrix which has been “functionalized,” such as by applying a layer or coating of an intermediate material including reactive groups which permit covalent attachment to molecules such as polynucleotides. Supports may include polyacrylamide hydrogel supported on an inert substrate such as glass. Molecules (e.g., polynucleotides) may be directly covalently attached to an intermediate material (e.g., a hydrogel). A support may include a plurality of particles or beads each having a different attached analyte.

As used herein, when an oligonucleotide or polynucleotide is described as “including” a nucleoside or nucleotide described herein, it includes when the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein may form a covalent bond with the oligonucleotide or polynucleotide. In one embodiment, the covalent bond is formed between a 3′ hydroxy group of the oligonucleotide or polynucleotide with the 5′ phosphate group of a nucleotide as a phosphodiester bond between the 3′ carbon atom of the oligonucleotide or polynucleotide and the 5′ carbon atom of the nucleotide.

As used herein, “derivative” or “analogue” means a synthetic nucleotide or nucleoside derivative having modified base moieties and/or modified sugar moieties. Such derivatives and analogs are discussed in, for example, Bucher, NUCLEOTIDE ANALOGS (John Wiley & Son, 1980) and Uhlmann et al., “Antisense Oligonucleotides: A New Therapeutic Principle,” Chemical Reviews 90:543-584 (1990), both of which are hereby incorporated by reference in their entirety. Nucleotide analogs may also include modified phosphodiester linkages, including phosphorothioate, phosphorodithioate, alkyl-phosphonate, phosphoranilidate and phosphoramidate linkages. “Derivative”, “analog”, and “modified” as used herein, may be used interchangeably, and are encompassed by the terms “nucleotide” and “nucleoside” as described herein.

As used herein, the term “phosphate” is used in its ordinary sense as understood by those skilled in the art, and includes its protonated forms. As used herein, the terms “monophosphate”, “diphosphate”, and “triphosphate” are used in their ordinary sense as understood by those skilled in the art, and include protonated forms. In the present disclosure, R₃ includes a linker including three or more phosphate groups.

The nucleosides or nucleotides described in accordance with the present disclosure include a purine or pyrimidine base and a ribose or deoxyribose sugar moiety which has a blocking group covalently attached thereto, for example at the 3′O position, which renders the molecules useful in techniques requiring blocking of the 3′-OH group to prevent incorporation of additional nucleotides, such as for example in sequencing reactions, polynucleotide synthesis, nucleic acid amplification, nucleic acid hybridization assays, single nucleotide polymorphism studies, and other such techniques.

Where the term “blocking group” is used herein in the context of the disclosure, this includes “Z” blocking groups described herein. However, it will be appreciated that, in the methods described and claimed herein, where mixtures of nucleotides are used, these may include the same type of blocking, i.e. “Z”-blocked. Where “Z”-blocked nucleotides are used, each “Z” group may be the same group, or not, if the detectable label forms part of the “Z” group (i.e. is not attached to the base).

Once the blocking group has been removed, it is possible to incorporate another nucleotide to the free 3′-OH group.

The molecule can be linked via the base to a detectable label by a desirable linker, which label may be a fluorophore, for example. The detectable label may instead, if desirable, be incorporated into the blocking groups of formula “Z.” The linker can be acid labile, photolabile or contain a disulfide linkage. Other linkages, in particular phosphine-cleavable azide-containing linkers, may be employed. Examples of labels and linkages include those disclosed in WO 03/048387, which is hereby incorporated by reference in its entirety. The term “hydroxy” as used herein includes a —OH group. R₂ as described herein may include a hydroxy (i.e., a —OH group) and/or R₂ as described herein may consist of —O—R₂ wherein R₂ is H or Z wherein Z is a removable protecting group comprising an azido group. In one embodiment, R₂ consists of —O—R₂ wherein R₂ is Z wherein Z is a removable protecting group comprising an azido group .

The terms “blocking group” and “blocking groups” as described herein refer to any atom or group of atoms that is added to a molecule in order to prevent existing groups in the molecule from undergoing unwanted chemical reactions. The phrases “blocking group” and “protecting group” may be used interchangeably. In order to ensure that only a single incorporation occurs, a structural modification (“blocking group” or “protecting group”) may be included in any labeled nucleotide that is added to a growing chain to ensure that only one nucleotide is incorporated. After a nucleotide with a blocking group has been added, the blocking group may then be removed, under reaction conditions which do not interfere with the integrity of the DNA being sequenced. The sequencing cycle can then continue with the incorporation of the next protected, labeled nucleotide.

To be useful in DNA sequencing, nucleotides, which are usually nucleotide triphosphates, may include a 3′-hydroxy blocking group so as to prevent the polymerase used to incorporate it into a polynucleotide chain from continuing to replicate once the base on the nucleotide is added. A blocking group should prevent additional nucleotide molecules from being added to the polynucleotide chain whilst simultaneously being easily removable from the sugar moiety without causing damage to the polynucleotide chain. Furthermore, the modified nucleotide may be compatible with the polymerase or another appropriate enzyme used to incorporate it into the polynucleotide chain. The ideal protecting group should exhibit long-term stability, be efficiently incorporated by the polymerase enzyme, cause blocking of secondary or further nucleotide incorporation, and have the ability to be removed under mild conditions that do not cause damage to the polynucleotide structure, preferably under aqueous conditions.

Examples of 3′ acetal blocking groups that may be useful in accordance with the present disclosure includes but are not limited to those described in U.S. application Ser. No. 16/724,088, which is hereby incorporated by reference in its entirety. Examples of azidomethyl blocking groups, which may be useful in accordance with the present disclosure, include but are not limited to acetal (e.g., 3′ acetal blocking groups or AOM) or thiocarbamate blocking groups which are described in are described in U.S. application Ser. No. 16/724,088, which is hereby incorporated by reference in its entirety. In one embodiment a 3′-OH blocking group will include moieties disclosed in WO2004/018497, which is hereby incorporated by reference in its entirety. The blocking group may, for example, be azidomethyl (CH₂N₃) or allyl.

In one embodiment, the 3′-hydroxy blocking group includes a reversible terminator. As described herein, examples of reversible terminator moieties are described, for example, in U.S. Pat Nos. 7,427,673, 7,414,116. and 7,057,026, as well as WO 91/06678 and WO 07/123744, each of which is incorporated herein by reference in its entirety. It will be understood that in some examples a nucleotide analog having a 3′ terminator moiety or lacking a 3′ hydroxyl (such as a dideoxynucleotide analog) can be used under conditions where the polynucleotide that has incorporated the nucleotide analog is not further extended. In some examples, the 3′-hydroxy blocking group may not include a reversible terminator moiety, or the 3′-hydroxy blocking group will not include a non-reversible terminator moiety, or the 3′-hydroxy blocking group will not include any terminator moiety at all. Reversible protecting groups have been described in, for example, Metzker et al., “Termination of DNA Synthesis by Novel 3′-modified-deoxyribonucleoside 5′-triphosphates,” Nucleic Acids Research 22(20):4259-426 (1994), which is hereby incorporated by reference in its entirety, and discloses the synthesis and use of eight 3′-modified 2-deoxyribonucleoside 5′-triphosphates (3′-modified dNTPs) and testing in two DNA template assays for incorporation activity. WO 2002/029003, which is hereby incorporated by reference in its entirety, describes a sequencing method which may include the use of an allyl protecting group to cap the 3′-OH group on a growing strand of DNA in a polymerase reaction. Examples of reversible terminators that may be useful with the methods described herein include but are not limited to an azidomethyl group, an acetal group, or a combination thereof.

In one embodiment, the method further includes removing the reversible terminator after the 3′ end of the complementary polynucleotide is covalently bonded to a phosphate group of the linker. The 3′ blocking group and fluorescent dye compounds can be removed (i.e., deprotected) simultaneously or sequentially to expose the nascent chain for further nucleotide incorporation. Typically, the identity of the incorporated nucleotide will be determined after each incorporation step, but this is not required. Similarly, U.S. Pat. No. 5,302,509, which is hereby incorporated by reference in its entirety, discloses a method to sequence polynucleotides immobilized on a solid support. The removal of the blocking group allows for further polymerization to occur.

This disclosure encompasses nucleotides including a fluorescent label that may be used in any method disclosed herein, on its own or incorporated into or associated with a larger molecular structure or conjugate. R₄ as described herein includes a fluorescent label. In this context, the fluorescent label (or any other detection tag that may be used) is moved away from the nucleobase to the 5′ terminal phosphate, thereby allowing for careful control of enzyme catalysis. Incorporation of the nucleotide in this manner as described herein results in the release of the detection tag completely, leaving behind scarless DNA.

The fluorescent label can include compounds selected from any known fluorescent species, for example rhodamines or cyanines. A fluorescent label as disclosed herein may be attached to any position on a nucleotide base, and may optionally include a linker. The function of the linker is generally to aid chemical attachment of the fluorescent label to the nucleotide. In particular embodiments Watson-Crick base pairing can still be carried out for the resulting analogue. A linker group may be used to covalently attach a dye to the nucleoside or nucleotide. A linker moiety may be of sufficient length to connect a nucleotide to a compound such that the compound does not significantly interfere with the overall binding and recognition of the nucleotide by a nucleic acid replication enzyme. Thus, the linker can also include a spacer unit. The spacer distances, for example, the nucleotide base from a cleavage site or label. The linker can be for example an alkyl chain optionally having one or more heteroatom replacements. The linker may contain amide or ester groups in order to facilitate chemical coupling reactions. The linker may be synthesized using click chemistry. The linker may contain triazole groups. The linker may contain other aryl groups.

As described herein, the present disclosure relates to sequencing chemistry which may enable the production of a scarless SBS. As disclosed herein, detection of a fluorescent signal may occur once the nucleotide and the polymerase are bound to the clustered DNA, opposite to the template strand, but prior to actual nucleotide incorporation (interchangeably referred to herein as, for example, a complexation condition, a non-incorporating condition, and a pause of catalysis). This aspect utilizes controlled catalysis in which the chemical incorporation of a nucleotide is either paused long enough or completely prevented in order to detect the signal and call the correct base during a complexation condition.

Stable binding of a nucleotide substrate carrying a fluorescent dye label by a polymerase-P/T complex on the surface of a flow cell may occur under varying conditions. After stable binding, excess nucleotide in solution may be washed away. As an example, the binding of the nucleotide substrate carrying a fluorescent dye label on the surface of a flow cell may occur under non-catalytic conditions. When non-catalytic conditions are maintained, the nucleotide-polymerase-P/T ternary complex may be stabilized and maintain the complexation condition as described herein. While the nitrogenous base is identified by its respective dye label, and, once signal detection (and thus base calling) has been achieved, the system may switch from non-incorporating conditions (i.e., the complexation condition as described herein), to incorporating conditions (i.e., the polymerization condition as described herein), by exchanging solutions.

Changes in conditions may facilitate the transition from complexation conditions (interchangeably referred to herein as, for example, a complexation condition and/or a non-incorporating condition) to polymerization conditions (interchangeably referred to herein as, for example, a polymerization condition, an incorporating condition, and/or a catalytic condition). In the presence of a catalytic condition, the DNA polymerase may incorporate the nucleotide to the DNA, causing dissociation of the leaving group (e.g., 5-prime polyphosphate of the nucleotide), which may carry with it the fluorescent label. In one embodiment, nucleotides that, in addition to the 5′ terminal phosphate modification, may contain a 3′ reversible terminator (e.g. AZM group), as currently used in traditional SBS. As described herein, this method promotes precise control of nucleotide incorporation, thereby enabling in each cycle the extension of a single nucleotide per DNA strand, particularly in further embodiments to be described below.

The complexation condition as described herein refers to a condition effective to form a complex but not effective to form polymerization. Detection of a fluorescent signal may occur once a free nucleotide and a polymerase are bound to complementary polynucleotide, opposite to the template polynucleotide, but prior to actual nucleotide incorporation (this complex that is formed prior to nucleotide incorporation is referred to herein as, for example, a complexation condition). A complexation condition as described herein may utilize controlled catalysis in which the incorporation of a nucleotide is either paused long enough or completely prevented in order to detect a signal and call a correct base. Thus, the contacting of a plurality of polymerases with a plurality of template polynucleotides and a plurality of free nucleotides, wherein at least one template polynucleotide is hybridized to a complementary polynucleotide, wherein each complementary polynucleotide includes a 3-prime end overhung by a 5-prime end of the template polynucleotide, in accordance with the present disclosure, may occur under a complexation condition. The complex formed during the complexation condition may include a polymerase, template polynucleotide, complementary polynucleotide, and one of a plurality of free nucleotides that is complementary to the most 3-prime nucleotide of the 5-prime end of the template polynucleotide overhanging the complementary polynucleotide.

This aspect utilizes controlled catalysis in which the chemical incorporation of a nucleotide is either paused long enough or completely prevented in order to detect the signal and call the correct base during a complexation condition. In one embodiment, the complexation condition includes a non-catalytic metal cation. Examples of non-catalytic metal cations as described herein include but are not limited to one or more of Ca²⁺, Zn²⁺, Co²⁺, Ni²⁺, Eu²⁺, Sr²⁺, Ba²⁺, Fe²⁺, Eu²⁺, and any combination thereof. The concentration of the non-catalytic metal cation present is less than or equal to about 100 mM. For example, the concentration of the non-catalytic metal may be about 100 mM, about 95 mM, about 90 mM, about 85 mM, about 80 mM, about 75 mM, about 70 mM, about 65 mM, about 60 mM, about 55 mM, about 50 mM, about 45 mM, about 40 mM, about 35 mM, about 30 mM, about 25 mM, about 20 mM, about 15 mM, about 10 mM, about 9 mM, about 8 mM, about 7 mM, about 6 mM, about 5 mM, about 4 mM, about 3 mM, about 2 mM, about 1 mM, less than 1 mM, or any amount therebetween. In one embodiment, the concentration of the non-catalytic metal cation present during the complexation condition may be less than or equal to about 10 mM.

In one embodiment, the complexation condition includes a chelating agent. Examples of chelating agent include but are not limited to ethylene glycol-bis(β-aminoethyl ether)-N,N,N′,N′-tetraacetic acid (EGTA), nitriloacetic acid, tetrasodium iminodisuccinate, ethylene glycol tetraacetic acid, polyaspartic acid, ethylenediamine-N,N′-disuccinic acid (EDDS), methylglycindiacetic acid (MGDA), and any combination thereof.

In one embodiment, the complexation condition further includes an inhibitor selected from the group consisting of a non-competitive inhibitor, a competitive inhibitor, and a combination thereof.

In one embodiment, the complexation condition includes a non-competitive inhibitor. The non-competitive inhibitor may be, for example, one or more of an aminoglycoside, a pyrophosphate analog, a melanin, a phosphonoacetate, a hypophosphate, and a rifamycin. Examples of non-competitive inhibitors that may be useful in the complexation condition of the present disclosure include but are not limited to Abacavir hemisulfate (reverse transcriptase inhibitor; antiretroviral); Actinomycin D (inhibits RNA polymerase); Acyclovir (inhibits viral DNA polymerase; antiherpetic agent); AM-TS23 (DNA polymerase λ and β inhibitor); α-Amanitin (inhibits RNA polymerase II); Aphidicolin (DNA polymerase α, δ and ε inhibitor); Azidothymidine (selective reverse transcriptase inhibitor; antiretroviral); BMH 21 (RNA polymerase 1 inhibitor; also p53 pathway activator); BMS 986094 (prodrug of HCV RNA polymerase inhibitor 2′-C-methyl guanosine triphosphate; potent HCV replication inhibitor); Delavirdine mesylate (non-nucleoside reverse transcriptase inhibitor); Entecavir (potent and selective hepatitis B virus inhibitor); Mithramycin A (inhibitor of DNA and RNA polymerase); Tenofovir (reverse transcriptase inhibitor); and Thiolutin (bacterial RNA polymerase inhibitor).

In one embodiment, the complexation condition includes a competitive inhibitor. Examples of competitive inhibitors that may be useful in the complexation condition of the present disclosure include but are not limited to aphidicolin, beta-D-arabinofuranosyl-CTP, amiloride, dehydroaltenusin, and any combination thereof.

When the complexation condition includes a non-catalytic metal, that non-catalytic metal may be selected from the group consisting of one or more of Ca2+, Zn2+, Co2+, Ni2+, Eu2+, Sr2+, Ba2+, Fe2+, and Eu2+. The concentration of the non-catalytic metal may be between 0 and 100 mM. For example, the concentration of the non-catalytic metal may be about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, and about 100 mM, or any amount therebetween. In some examples, the concentration of the non-catalytic metal is between about 0.1 mM and about 10 mM, or between about 1 mM and about 10 mM. In one embodiment, the concentration of the non-catalytic metal is up to about 10 mM. In one embodiment, a non-catalytic metal is required to maintain the complexation condition.

The pH may also be set to facilitate and/or maintain complexation conditions. In one embodiment, the complexation condition includes a pH that is less than about 6. The pH may be, for example about 5, about 4, about 3, about 2, about 1, or less than 1.

In one embodiment, the complexation condition includes a solvent additive. Examples of solvent additives that may be useful in the complexation condition of the present disclosure include but are not limited to ethanol, methanol, tetrahydrofuran, dioxane, dimethylamine, dimethylformamide, dimethyl sulfoxide, lithium, L-cysteine, and a combination thereof. In one embodiment, the complexation condition includes deuterium.

Changes in conditions may facilitate the transition from a complexation condition to a polymerization condition. A polymerization condition as described herein promotes the formation of a complex that allows for incorporated of a nucleotide onto the 3-prime end of the complementary polynucleotide by the polymerase of the complex. The transition from a complexation condition (also referred to herein as non-incorporating condition) to a polymerization condition (also referred to herein as incorporating condition) may be achieved by, for example, switching from non-catalytic to catalytic conditions, so that the DNA polymerase may incorporate a nucleotide to the DNA, thereby causing dissociation of a leaving group which may carry with it a fluorescent dye attached thereto. The polymerization step may be allowed to proceed for a time sufficient to allow incorporation of a nucleotide.

Polymerase in accordance with the present disclosure may include any polymerase that can tolerate incorporation of a phosphate-labeled nucleotide. Examples of polymerases that may be useful in accordance with the present disclosure include but are not limited to phi29 polymerase, a klenow fragment, DNA polymerase I, DNA polymerase III, GA-1, PZA, phi15, Nf, G1, PZE, PRD1, B103, GA-1, 9oN polymerase, Bst, Bsu, T4, T5, T7, Taq, Vent, RT, pol beta, and pol gamma. Polymerases engineered to have specific properties may also be used.

The polymerization condition may include various concentrations of Mg²⁺ ions and/or Mn²⁺ ions. For example, the concentration of the Mg²⁺ ions may be about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, and about 100 mM, or any amount therebetween. Similarly, the concentration of the Mn²⁺ ions may be about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, and about 100 mM, or any amount therebetween. In one embodiment, when the polymerization condition includes a concentration of Mg²⁺ ions, the concentration of Mg²⁺ ions may be in a range of about 0.1 mM to about 10 mM, or a concentration of Mn²⁺ ions, the concentration of Mn²⁺ ions may be in a range of about 0.1 mM to about 10 mM.

The pH may also be adjusted to facilitate polymerization conditions. In one embodiment, the polymerization condition includes a pH that is greater than or equal to about 6. The pH may be, for example about 6, about 7, about 8, about 9, about 10, about 11, about 12, about 13, or about 14.

The steps of (a) contacting a polymerase with a template polynucleotide and a plurality of free nucleotides, wherein the template polynucleotide is hybridized to a complementary polynucleotide including a 3′ end overhung by a 5′ terminal fragment of the template polynucleotide, and the plurality of free nucleotides include a compound of Formula (I), where the contacting occurs under a complexation condition, the complexation condition effective to form a complex but not effective to form polymerization, where the complex includes the polymerase, the template polynucleotide, the complementary polynucleotide, and one of the plurality of free nucleotides that is complementary to a first nucleotide of the 5′ terminal fragment of the template polynucleotide; (b) detecting a signal from the fluorescent label; and (c) exposing the complex to a polymerization condition may be repeated one or more times.

The free nucleotide, in one embodiment, may further includes a non-bridging thiol or a bridging nitrogen. Generally, a non-bridging thiol of a nucleotide may include a thiol substituted for a carbonyl oxygen in a phosphodiester bond between 5′ phosphate groups of a nucleotide, such as in the following example:

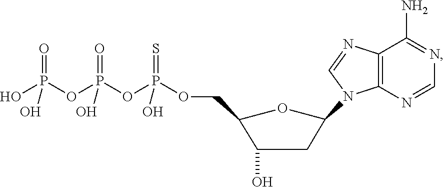 [SMILES: Nc1ncnc2c1ncn2[C@H]1C[C@H](O)[C@@H](COP(O)(=S)OP(=O)(O)OP(=O)(O)O)O1]

with further modifications of a free nucleotide in accordance with other aspects of this disclosure. And generally, a bridging nitrogen may include a nitrogen substituted for an oxygen in an ether of a phosphodiester bond between 5′ phosphate groups of a nucleotide, such as in the following example:

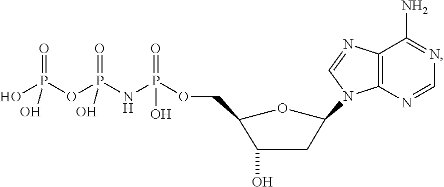 [SMILES: Nc1ncnc2c1ncn2[C@H]1C[C@H](O)[C@@H](COP(=O)(O)NP(=O)(O)OP(=O)(O)O)O1]

with further modifications of a free nucleotide in accordance with other aspects of this disclosure.

The polymerase may, in one embodiment, include a mutation. In one embodiment, the mutation modifies speed of (a) contacting a polymerase with a template polynucleotide and a plurality of free nucleotides, where the template polynucleotide is hybridized to a complementary polynucleotide including a 3′ end overhung by a 5′ terminal fragment of the template polynucleotide, and the plurality of free nucleotides include a compound of Formula (I), where the contacting occurs under a complexation condition, the complexation condition effective to form a complex but not effective to form polymerization, where the complex includes the polymerase, the template polynucleotide, the complementary polynucleotide, and one of the plurality of free nucleotides that is complementary to a first nucleotide of the 5′ terminal fragment of the template polynucleotide; and/or (b) detecting a signal from the fluorescent label; and/or (c) exposing the complex to a polymerization condition may be repeated one or more times.

As described, each nucleotide may be brought into contact with a target sequentially, with removal of non-incorporated nucleotides prior to addition of the next nucleotide, where detection and removal of the label and the blocking group may be carried out either after addition of each nucleotide, or after addition of all four nucleotides.

All of the nucleotides may be brought into contact with a target simultaneously, i.e., a composition comprising all of the different nucleotides may be brought into contact with a target, and non-incorporated nucleotides may be removed prior to detection and subsequent to removal of the label and the blocking group.

### Library Preparation

Libraries including polynucleotides may be prepared in any suitable manner to attach oligonucleotide adapters to target polynucleotides. As used herein, a “library” is a population of polynucleotides from a given source or sample. A library includes a plurality of target polynucleotides. As used herein, a “target polynucleotide” is a polynucleotide that is desired to sequence. The target polynucleotide may be essentially any polynucleotide of known or unknown sequence. It may be, for example, a fragment of genomic DNA or cDNA. Sequencing may result in determination of the sequence of the whole, or a part of the target polynucleotides. The target polynucleotides may be derived from a primary polynucleotide sample that has been randomly fragmented. The target polynucleotides may be processed into templates suitable for amplification by the placement of universal primer sequences at the ends of each target fragment. The target polynucleotides may also be obtained from a primary RNA sample by reverse transcription into cDNA.

As used herein, the terms “polynucleotide” and “oligonucleotide” may be used interchangeably and refer to a molecule including two or more nucleotide monomers covalently bound to one another, typically through a phosphodiester bond. Polynucleotides typically contain more nucleotides than oligonucleotides. For purposes of illustration and not limitation, a polynucleotide may be considered to contain 15, 20, 30, 40, 50, 100, 200, 300, 400, 500, or more nucleotides, while an oligonucleotide may be considered to contain 100, 50, 20, 15 or less nucleotides.

Polynucleotides and oligonucleotides may include deoxyribonucleic acid (DNA) or ribonucleic acid (RNA). The terms should be understood to include, as equivalents, analogs of either DNA or RNA made from nucleotide analogs and to be applicable to single stranded (such as sense or antisense) and double stranded polynucleotides. The term as used herein also encompasses cDNA, that is complementary or copy DNA produced from an RNA template, for example by the action of reverse transcriptase.

Primary polynucleotide molecules may originate in double-stranded DNA (dsDNA) form (e.g. genomic DNA fragments, PCR and amplification products and the like) or may have originated in single-stranded form, as DNA or RNA, and been converted to dsDNA form. By way of example, mRNA molecules may be copied into double-stranded cDNAs using standard techniques well known in the art. The precise sequence of primary polynucleotides is generally not material to the disclosure presented herein, and may be known or unknown.

In some embodiments, the primary target polynucleotides are RNA molecules. In an aspect of such embodiments, RNA isolated from specific samples is first converted to double-stranded DNA using techniques known in the art. The double-stranded DNA may then be index tagged with a library specific tag. Different preparations of such double-stranded DNA including library specific index tags may be generated, in parallel, from RNA isolated from different sources or samples. Subsequently, different preparations of double-stranded DNA including different library specific index tags may be mixed, sequenced en masse, and the identity of each sequenced fragment determined with respect to the library from which it was isolated/derived by virtue of the presence of a library specific index tag sequence.

In some embodiments, the primary target polynucleotides are DNA molecules. For example, the primary polynucleotides may represent the entire genetic complement of an organism, and are genomic DNA molecules, such as human DNA molecules, which include both intron and exon sequences (coding sequence), as well as non-coding regulatory sequences such as promoter and enhancer sequences. Although it could be envisaged that particular sub-sets of polynucleotide sequences or genomic DNA could also be used, such as, for example, particular chromosomes or a portion thereof. In many embodiments, the sequence of the primary polynucleotides is not known. The DNA target polynucleotides may be treated chemically or enzymatically either prior to, or subsequent to a fragmentation processes, such as a random fragmentation process, and prior to, during, or subsequent to the ligation of the adapter oligonucleotides.

Preferably, the primary target polynucleotides are fragmented to appropriate lengths suitable for sequencing. The target polynucleotides may be fragmented in any suitable manner. Preferably, the target polynucleotides are randomly fragmented. Random fragmentation refers to the fragmentation of a polynucleotide in a non-ordered fashion by, for example, enzymatic, chemical or mechanical means. Such fragmentation methods are known in the art and utilize standard methods (Sambrook and Russell, Molecular Cloning, A Laboratory Manual, third edition, which is hereby incorporated by reference in its entirety). For the sake of clarity, generating smaller fragments of a larger piece of polynucleotide via specific PCR amplification of such smaller fragments is not equivalent to fragmenting the larger piece of polynucleotide because the larger piece of polynucleotide remains in intact (i.e., is not fragmented by the PCR amplification). Moreover, random fragmentation is designed to produce fragments irrespective of the sequence identity or position of nucleotides including and/or surrounding the break.

In some embodiments, the random fragmentation is by mechanical means such as nebulization or sonication to produce fragments of about 50 base pairs in length to about 1500 base pairs in length, such as 50-700 base pairs in length or 50-500 base pairs in length.

Fragmentation of polynucleotide molecules by mechanical means (nebulization, sonication and Hydroshear for example) may result in fragments with a heterogeneous mix of blunt and 3′- and 5′-overhanging ends. Fragment ends may be repaired using methods or kits (such as the Lucigen DNA terminator End Repair Kit) known in the art to generate ends that are optimal for insertion, for example, into blunt sites of cloning vectors. In some embodiments, the fragment ends of the population of nucleic acids are blunt ended. The fragment ends may be blunt ended and phosphorylated. The phosphate moiety may be introduced via enzymatic treatment, for example, using polynucleotide kinase.

In some embodiments, the target polynucleotide sequences are prepared with single overhanging nucleotides by, for example, activity of certain types of DNA polymerase such as Taq polymerase or Klenow exo minus polymerase which has a nontemplate-dependent terminal transferase activity that adds a single deoxynucleotide, for example, deoxyadenosine (A) to the 3′ ends of, for example, PCR products. Such enzymes may be utilized to add a single nucleotide ‘A’ to the blunt ended 3′ terminus of each strand of the target polynucleotide duplexes. Thus, an ‘A’ could be added to the 3′ terminus of each end repaired duplex strand of the target polynucleotide duplex by reaction with Taq or Klenow exo minus polymerase, while the adapter polynucleotide construct could be a T-construct with a compatible ‘T’ overhang present on the 3′ terminus of each duplex region of the adapter construct. This end modification also prevents self-ligation of the target polynucleotides such that there is a bias towards formation of the combined ligated adapter-target polynucleotides.

In some embodiments, fragmentation is accomplished through tagmentation as described in, for example, WO 2016/130704, which is hereby incorporated by reference in its entirety. In such methods transposases are employed to fragment a double stranded polynucleotide and attach a universal primer sequence into one strand of the double stranded polynucleotide. The resulting molecule may be gap-filled and subject to extension, for example by PCR amplification, using primers that include a 3′ end having a sequence complementary to the attached universal primer sequence and a 5′ end that contains other sequences of an adapter.

The adapters may be attached to the target polynucleotide in any other suitable manner. In some embodiments, the adapters are introduced in a multi-step process, such as a two-step process, involving ligation of a portion of the adapter to the target polynucleotide having a universal primer sequence. The second step includes extension, for example by PCR amplification, using primers that include a 3′ end having a sequence complementary to the attached universal primer sequence and a 5′ end that contains other sequences of an adapter. By way of example, such extension may be performed as described in U.S. Pat. No. 8,053,192, which is hereby incorporated by reference in its entirety. Additional extensions may be performed to provide additional sequences to the 5′ end of the resulting previously extended polynucleotide.

In some embodiments, the entire adapter is ligated to the fragmented target polynucleotide. Preferably, the ligated adapter includes a double stranded region that is ligated to a double stranded target polynucleotide. Preferably, the double-stranded region is as short as possible without loss of function. In this context, “function” refers to the ability of the double-stranded region to form a stable duplex under standard reaction conditions. In some embodiments, standard reactions conditions refer to reaction conditions for an enzyme-catalyzed polynucleotide ligation reaction, which will be well known to the skilled reader (e.g. incubation at a temperature in the range of 4° C. to 25° C. in a ligation buffer appropriate for the enzyme), such that the two strands forming the adapter remain partially annealed during ligation of the adapter to a target molecule. Ligation methods are known in the art and may utilize standard methods (Sambrook and Russell, Molecular Cloning, A Laboratory Manual, third edition, which is hereby incorporated by reference in its entirety). Such methods utilize ligase enzymes such as DNA ligase to effect or catalyze joining of the ends of the two polynucleotide strands of, in this case, the adapter duplex oligonucleotide and the target polynucleotide duplexes, such that covalent linkages are formed. The adapter duplex oligonucleotide may contain a 5′-phosphate moiety in order to facilitate ligation to a target polynucleotide 3′-OH. The target polynucleotide may contain a 5′-phosphate moiety, either residual from the shearing process, or added using an enzymatic treatment step, and has been end repaired, and optionally extended by an overhanging base or bases, to give a 3′-OH suitable for ligation. In this context, attaching means covalent linkage of polynucleotide strands which were not previously covalently linked. In a particular aspect of the disclosure, such attaching takes place by formation of a phosphodiester linkage between the two polynucleotide strands, but other means of covalent linkage (e.g. non-phosphodiester backbone linkages) may be used. Ligation of adapters to target polynucleotides is described in more detail in, for example, U.S. Pat. No. 8,053,192, which is hereby incorporated by reference in its entirety.

Any suitable adapter may be attached to a target polynucleotide via any suitable process, such as those discussed above. The adapter includes a library-specific index tag sequence. The index tag sequence may be attached to the target polynucleotides from each library before the sample is immobilized for sequencing. The index tag is not itself formed by part of the target polynucleotide, but becomes part of the template for amplification. The index tag may be a synthetic sequence of nucleotides which is added to the target as part of the template preparation step. Accordingly, a library-specific index tag is a nucleic acid sequence tag which is attached to each of the target molecules of a particular library, the presence of which is indicative of or is used to identify the library from which the target molecules were isolated.

Preferably, the index tag sequence is 20 nucleotides or less in length. For example, the index tag sequence may be 1-10 nucleotides or 4-6 nucleotides in length. A four nucleotide index tag gives a possibility of multiplexing 256 samples on the same array, a six base index tag enables 4,096 samples to be processed on the same array.

The adapters may contain more than one index tag so that the multiplexing possibilities may be increased.

The adapters preferably include a double stranded region and a region including two non-complementary single strands. The double-stranded region of the adapter may be of any suitable number of base pairs. Preferably, the double stranded region is a short double-stranded region, typically including 5 or more consecutive base pairs, formed by annealing of two partially complementary polynucleotide strands. This “double-stranded region” of the adapter refers to a region in which the two strands are annealed and does not imply any particular structural conformation. In some embodiments, the double stranded region includes 20 or less consecutive base pairs, such as 10 or less or 5 or less consecutive base pairs.

The stability of the double-stranded region may be increased, and hence its length potentially reduced, by the inclusion of non-natural nucleotides which exhibit stronger base-pairing than standard Watson-Crick base pairs. Preferably, the two strands of the adapter are 100% complementary in the double-stranded region.

When the adapter is attached to the target polynucleotide, the non-complementary single stranded region may form the 5′ and 3′ ends of the polynucleotide to be sequenced. The term “non-complementary single stranded region” refers to a region of the adapter where the sequences of the two polynucleotide strands forming the adapter exhibit a degree of non-complementarity such that the two strands are not capable of fully annealing to each other under standard annealing conditions for a PCR reaction.

The non-complementary single stranded region is provided by different portions of the same two polynucleotide strands which form the double-stranded region. The lower limit on the length of the single-stranded portion will typically be determined by function of, for example, providing a suitable sequence for binding of a primer for primer extension, PCR and/or sequencing. Theoretically there is no upper limit on the length of the unmatched region, except that in general it is advantageous to minimize the overall length of the adapter, for example, in order to facilitate separation of unbound adapters from adapter-target constructs following the attachment step or steps. Therefore, it is generally preferred that the non-complementary single-stranded region of the adapter is 50 or less consecutive nucleotides in length, such as 40 or less, 30 or less, or 25 or less consecutive nucleotides in length.

The library-specific index tag sequence may be located in a single-stranded, double-stranded region, or span the single-stranded and double-stranded regions of the adapter. Preferably, the index tag sequence is in a single-stranded region of the adapter.

The adapters may include any other suitable sequence in addition to the index tag sequence. For example, the adapters may include universal extension primer sequences, which are typically located at the 5′ or 3′ end of the adapter and the resulting polynucleotide for sequencing. The universal extension primer sequences may hybridize to complementary primers bound to a surface of a solid substrate. The complementary primers include a free 3′ end from which a polymerase or other suitable enzyme may add nucleotides to extend the sequence using the hybridized library polynucleotide as a template, resulting in a reverse strand of the library polynucleotide being coupled to the solid surface. Such extension may be part of a sequencing run or cluster amplification.

In some embodiments, the adapters include one or more universal sequencing primer sequences. The universal sequencing primer sequences may bind to sequencing primers to allow sequencing of an index tag sequence, a target sequence, or an index tag sequence and a target sequence.

The precise nucleotide sequence of the adapters is generally not material to the disclosure and may be selected by the user such that the desired sequence elements are ultimately included in the common sequences of the library of templates derived from the adapters to, for example, provide binding sites for particular sets of universal extension primers and/or sequencing primers.

The adapter oligonucleotides may contain exonuclease resistant modifications such as phosphorothioate linkages.

Preferably, the adapter is attached to both ends of a target polypeptide to produce a polynucleotide having a first adapter-target-second adapter sequence of nucleotides. The first and second adapters may be the same or different. Preferably, the first and second adapters are the same. If the first and second adapters are different, at least one of the first and second adapters includes a library-specific index tag sequence.

It will be understood that a “first adapter-target-second adapter sequence” or an “adapter-target-adapter” sequence refers to the orientation of the adapters relative to one another and to the target and does not necessarily mean that the sequence may not include additional sequences, such as linker sequences, for example.

Other libraries may be prepared in a similar manner, each including at least one library-specific index tag sequence or combinations of index tag sequences different than an index tag sequence or combination of index tag sequences from the other libraries.

As used herein, “attached” or “bound” are used interchangeably in the context of an adapter relative to a target sequence. As described above, any suitable process may be used to attach an adapter to a target polynucleotide. For example, the adapter may be attached to the target through ligation with a ligase; through a combination of ligation of a portion of an adapter and addition of further or remaining portions of the adapter through extension, such as PCR, with primers containing the further or remaining portions of the adapters; trough transposition to incorporate a portion of an adapter and addition of further or remaining portions of the adapter through extension, such as PCR, with primers containing the further or remaining portions of the adapters; or the like. Preferably, the attached adapter oligonucleotide is covalently bound to the target polynucleotide.

After the adapters are attached to the target polynucleotides, the resulting polynucleotides may be subjected to a clean-up process to enhance the purity to the adapter-target-adapter polynucleotides by removing at least a portion of the unincorporated adapters. Any suitable clean-up process may be used, such as electrophoresis, size exclusion chromatography, or the like. In some embodiments, solid phase reverse immobilization (SPRI) paramagnetic beads may be employed to separate the adapter-target-adapter polynucleotides from the unattached adapters. While such processes may enhance the purity of the resulting adapter-target-adapter polynucleotides, some unattached adapter oligonucleotides likely remain.

### Preparation of Immobilized Samples for Sequencing

In accordance with the present disclosure, a plurality of adapter-target-adapter polynucleotide molecules from one or more sources are then immobilized and amplified prior to sequencing. Methods for attaching adapter-target-adapter molecules from one or more sources to a substrate are known in the art. Likewise, methods for amplifying immobilized adapter-target-adapter molecules include, but are not limited to, bridge amplification and kinetic exclusion. Methods for immobilizing and amplifying prior to sequencing are described in, for instance, U.S. Pat. No. 8,053,192, WO 2016/130704, U.S. Pat. No. 8,895,249, and U.S. Pat. No. 9,309,502, all of which are hereby incorporated by reference in their entirety.

A sample, including pooled samples, can then be immobilized in preparation for sequencing. Sequencing can be performed as an array of single molecules, or can be amplified prior to sequencing. The amplification can be carried out using one or more immobilized primers. The immobilized primer(s) can be a lawn on a planar surface, or on a pool of beads. The pool of beads can be isolated into an emulsion with a single bead in each “compartment” of the emulsion. At a concentration of only one template per “compartment”, only a single template is amplified on each bead.

The term “solid-phase amplification” as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Solid phase PCR covers systems such as emulsions, wherein one primer is anchored to a bead and the other is in free solution, and colony formation in solid phase gel matrices wherein one primer is anchored to the surface, and one is in free solution.

In some embodiments, the solid support includes a patterned surface. A “patterned surface” refers to an arrangement of different regions in or on an exposed layer of a solid support. For example, one or more of the regions can be features where one or more amplification primers are present. The features can be separated by interstitial regions where amplification primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Pat. Nos. 8,778,848; 8,778,849; and 9,079,148, and U.S. Pat. Publ. No. 2014/0243224, each of which is incorporated herein by reference in its entirety.

In some embodiments, the solid support includes an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface can be wells in an array of wells (e.g. microwells or nanowells) on glass, silicon, plastic or other suitable solid supports with patterned, covalently-linked gel such as poly(N-(5-azidoacetamidylpentyl)acrylamide-co-acrylamide) (PAZAM, see, for example, U.S. Pat. Publ. No. 2013/184796, WO 2016/066586, and WO 2015/002813, each of which is incorporated herein by reference in its entirety). The process creates gel pads used for sequencing that can be stable over sequencing runs with a large number of cycles. The covalent linking of the polymer to the wells is helpful for maintaining the gel in the structured features throughout the lifetime of the structured substrate during a variety of uses. However in many embodiments, the gel need not be covalently linked to the wells. For example, in some conditions, silane free acrylamide (SFA, see, for example, U.S. Pat. No. 8,563,477, which is incorporated herein by reference in its entirety) which is not covalently attached to any part of the structured substrate, can be used as the gel material.

In particular embodiments, a structured substrate can be made by patterning a solid support material with wells (e.g. microwells or nanowells), coating the patterned support with a gel material (e.g. PAZAM, SFA or chemically modified variants thereof, such as the azidolyzed version of SFA (azido-SFA)) and polishing the gel coated support, for example via chemical or mechanical polishing, thereby retaining gel in the wells but removing or inactivating substantially all of the gel from the interstitial regions on the surface of the structured substrate between the wells. Primer nucleic acids can be attached to gel material. A solution of target nucleic acids (e.g. a fragmented human genome) can then be contacted with the polished substrate such that individual target nucleic acids will seed individual wells via interactions with primers attached to the gel material; however, the target nucleic acids will not occupy the interstitial regions due to absence or inactivity of the gel material. Amplification of the target nucleic acids will be confined to the wells since absence or inactivity of gel in the interstitial regions prevents outward migration of the growing nucleic acid colony. The process is conveniently manufacturable, being scalable and utilizing conventional micro- or nanofabrication methods.

Although the disclosure encompasses “solid-phase” amplification methods in which only one amplification primer is immobilized (the other primer usually being present in free solution), it is preferred for the solid support to be provided with both the forward and the reverse primers immobilized. In practice, there will be a ‘plurality’ of identical forward primers and/or a ‘plurality’ of identical reverse primers immobilized on the solid support, since the amplification process requires an excess of primers to sustain amplification. References herein to forward and reverse primers are to be interpreted accordingly as encompassing a ‘plurality’ of such primers unless the context indicates otherwise.

As will be appreciated by the skilled reader, any given amplification reaction requires at least one type of forward primer and at least one type of reverse primer specific for the template to be amplified. However, in certain embodiments the forward and reverse primers may include template-specific portions of identical sequence, and may have entirely identical nucleotide sequence and structure (including any non-nucleotide modifications). In other words, it is possible to carry out solid-phase amplification using only one type of primer, and such single-primer methods are encompassed within the scope of the disclosure. Other embodiments may use forward and reverse primers which contain identical template-specific sequences but which differ in some other structural features. For example one type of primer may contain a non-nucleotide modification which is not present in the other.

In all embodiments of the disclosure, primers for solid-phase amplification are preferably immobilized by single point covalent attachment to the solid support at or near the 5′ end of the primer, leaving the template-specific portion of the primer free to anneal to its cognate template and the 3′ hydroxyl group free for primer extension. Any suitable covalent attachment means known in the art may be used for this purpose. The chosen attachment chemistry will depend on the nature of the solid support, and any derivatization or functionalization applied to it. The primer itself may include a moiety, which may be a non-nucleotide chemical modification, to facilitate attachment. In a particular embodiment, the primer may include a sulphur-containing nucleophile, such as phosphorothioate or thiophosphate, at the 5′ end. In the case of solid-supported polyacrylamide hydrogels, this nucleophile will bind to a bromoacetamide group present in the hydrogel. A more particular means of attaching primers and templates to a solid support is via 5′ phosphorothioate attachment to a hydrogel including polymerized acrylamide and N-(5-bromoacetamidylpentyl) acrylamide (BRAPA), as described fully in WO 05/065814, which is hereby incorporated by reference in its entirety.

Certain embodiments of the disclosure may make use of solid supports including an inert substrate or matrix (e.g. glass slides, polymer beads, etc.) which has been “functionalized”, for example by application of a layer or coating of an intermediate material including reactive groups which permit covalent attachment to biomolecules, such as polynucleotides. Examples of such supports include, but are not limited to, polyacrylamide hydrogels supported on an inert substrate such as glass. In such embodiments, the biomolecules (e.g. polynucleotides) may be directly covalently attached to the intermediate material (e.g. the hydrogel), but the intermediate material may itself be non-covalently attached to the substrate or matrix (e.g. the glass substrate). The term “covalent attachment to a solid support” is to be interpreted accordingly as encompassing this type of arrangement.

The pooled samples may be amplified on beads wherein each bead contains a forward and reverse amplification primer. In a particular embodiment, the library of templates prepared according to the aspects of the present disclosure is used to prepare clustered arrays of nucleic acid colonies, analogous to those described in U.S. Pat. Publ. No. 2005/0100900, U.S. Pat. No. 7,115,400, WO 00/18957, and WO 98/44151, each of which is hereby incorporated by reference in its entirety, by solid-phase amplification and more particularly solid phase isothermal amplification. The terms ‘cluster’ and ‘colony’ are used interchangeably herein to refer to a discrete site on a solid support including a plurality of identical immobilized nucleic acid strands and a plurality of identical immobilized complementary nucleic acid strands. The term “clustered array” refers to an array formed from such clusters or colonies. In this context the term “array” is not to be understood as requiring an ordered arrangement of clusters.

The term “solid phase”, or “surface”, is used to mean either a planar array wherein primers are attached to a flat surface, for example, glass, silica or plastic microscope slides or similar flow cell devices; beads, wherein either one or two primers are attached to the beads and the beads are amplified; or an array of beads on a surface after the beads have been amplified.

Clustered arrays can be prepared using either a process of thermocycling, as described in WO 98/44151, which is hereby incorporated by reference in its entirety, or a process whereby the temperature is maintained as a constant, and the cycles of extension and denaturing are performed using changes of reagents. Such isothermal amplification methods are described in WO 02/46456 and U.S. Pat. Publ. No. 2008/0009420, which are hereby incorporated by reference in their entirety.

It will be appreciated that any of the amplification methodologies described herein or generally known in the art may be utilized with universal or target-specific primers to amplify immobilized DNA fragments. Suitable methods for amplification include, but are not limited to, the polymerase chain reaction (PCR), strand displacement amplification (SDA), transcription mediated amplification (TMA) and nucleic acid sequence based amplification (NASBA), as described in U.S. Pat. No. 8,003,354, which is incorporated herein by reference in its entirety. The above amplification methods may be employed to amplify one or more nucleic acids of interest. For example, PCR, including multiplex PCR, SDA, TMA, NASBA and the like may be utilized to amplify immobilized DNA fragments. In some embodiments, primers directed specifically to the polynucleotide of interest are included in the amplification reaction.

Other suitable methods for amplification of polynucleotides may include oligonucleotide extension and ligation, rolling circle amplification (RCA) (Lizardi et al., “Mutation Detection and Single-Molecule Counting Using Isothermal Rolling-Circle Amplification,” Nat. Genet. 19:225-232 (1998), which is hereby incorporated by reference in its entirety) and oligonucleotide ligation assay (OLA) (see generally U.S. Pat. Nos. 7,582,420, 5,185,243, 5,679,524, and 5,573,907; EP 0 320 308 B1; EP 0 336 731 B1; EP 0 439 182 B1; WO 90/01069; WO 89/12696; and WO 89/09835, all of which are hereby incorporated by reference in their entirety) technologies. It will be appreciated that these amplification methodologies may be designed to amplify immobilized DNA fragments. For example, in some embodiments, the amplification method may include ligation probe amplification or oligonucleotide ligation assay (OLA) reactions that contain primers directed specifically to the nucleic acid of interest. In some embodiments, the amplification method may include a primer extension-ligation reaction that contains primers directed specifically to the nucleic acid of interest. As a non-limiting example of primer extension and ligation primers that may be specifically designed to amplify a nucleic acid of interest, the amplification may include primers used for the GoldenGate assay (Illumina, Inc., San Diego, Calif.) as exemplified by U.S. Pat. Nos. 7,582,420 and 7,611,869, both of which are hereby incorporated by reference in their entirety.

Exemplary isothermal amplification methods that may be used in a method of the present disclosure include, but are not limited to, Multiple Displacement Amplification (MDA) as exemplified by, for example Dean et al., “Comprehensive Human Genome Amplification Using Multiple Displacement Amplification,” Proc. Natl. Acad. Sci. USA 99:5261-66 (2002), which is hereby incorporated by reference in its entirety, or isothermal strand displacement nucleic acid amplification exemplified by, for example U.S. Pat. No. 6,214,587, which is hereby incorporated by reference in its entirety. Other non-PCR-based methods that may be used in the present disclosure include, for example, strand displacement amplification (SDA) which is described in, for example Walker et al., Molecular Methods for Virus Detection (Academic Press, Inc., 1995); U.S. Pat. Nos. 5,455,166 and 5,130,238, and Walker et al., “Strand Displacement Amplification—An Isothermal, in Vitro DNA Amplification Technique,” Nucl. Acids Res. 20:1691-96 (1992), all of which are hereby incorporated by reference in their entirety, or hyper-branched strand displacement amplification which is described in, for example Lage et al., “Whole Genome Analysis of Genetic Alterations in Small DNA Samples Using Hyperbranched Strand Displacement Amplification and array-CGH,” Genome Res. 13:294-307 (2003), which is hereby incorporated by reference in its entirety. Isothermal amplification methods may be used with the strand-displacing Phi 29 polymerase or Bst DNA polymerase large fragment, 5′→3′ exo- for random primer amplification of genomic DNA. The use of these polymerases takes advantage of their high processivity and strand displacing activity. High processivity allows the polymerases to produce fragments that are 10-20 kb in length. As set forth above, smaller fragments may be produced under isothermal conditions using polymerases having low processivity and strand-displacing activity such as Klenow polymerase. Additional description of amplification reactions, conditions and components are set forth in detail in the disclosure of U.S. Pat. No. 7,670,810, which is incorporated herein by reference in its entirety.

Another polynucleotide amplification method that is useful in the present disclosure is Tagged PCR which uses a population of two-domain primers having a constant 5′ region followed by a random 3′ region as described, for example, in Grothues et al., “PCR Amplification of Megabase DNA With Tagged Random Primers (T-PCR),” Nucleic Acids Res. 21(5):1321-2 (1993), which is hereby incorporated by reference in its entirety. The first rounds of amplification are carried out to allow a multitude of initiations on heat denatured DNA based on individual hybridization from the randomly-synthesized 3′ region. Due to the nature of the 3′ region, the sites of initiation are contemplated to be random throughout the genome. Thereafter, the unbound primers may be removed and further replication may take place using primers complementary to the constant 5′ region.

In some embodiments, isothermal amplification can be performed using kinetic exclusion amplification (KEA), also referred to as exclusion amplification (ExAmp). A nucleic acid library of the present disclosure can be made using a method that includes a step of reacting an amplification reagent to produce a plurality of amplification sites that each includes a substantially clonal population of amplicons from an individual target nucleic acid that has seeded the site. In some embodiments the amplification reaction proceeds until a sufficient number of amplicons are generated to fill the capacity of the respective amplification site. Filling an already seeded site to capacity in this way inhibits target nucleic acids from landing and amplifying at the site thereby producing a clonal population of amplicons at the site. In some embodiments, apparent clonality can be achieved even if an amplification site is not filled to capacity prior to a second target nucleic acid arriving at the site. Under some conditions, amplification of a first target nucleic acid can proceed to a point that a sufficient number of copies are made to effectively outcompete or overwhelm production of copies from a second target nucleic acid that is transported to the site. For example in an embodiment that uses a bridge amplification process on a circular feature that is smaller than 500 nm in diameter, it has been determined that after 14 cycles of exponential amplification for a first target nucleic acid, contamination from a second target nucleic acid at the same site will produce an insufficient number of contaminating amplicons to adversely impact sequencing-by-synthesis analysis on an Illumina sequencing platform.

Amplification sites in an array can be, but need not be, entirely clonal in particular embodiments. Rather, for some applications, an individual amplification site can be predominantly populated with amplicons from a first target nucleic acid and can also have a low level of contaminating amplicons from a second target nucleic acid. An array can have one or more amplification sites that have a low level of contaminating amplicons so long as the level of contamination does not have an unacceptable impact on a subsequent use of the array. For example, when the array is to be used in a detection application, an acceptable level of contamination would be a level that does not impact signal to noise or resolution of the detection technique in an unacceptable way. Accordingly, apparent clonality will generally be relevant to a particular use or application of an array made by the methods set forth herein. Exemplary levels of contamination that can be acceptable at an individual amplification site for particular applications include, but are not limited to, at most 0.1%, 0.5%, 1%, 5%, 10% or 25% contaminating amplicons. An array can include one or more amplification sites having these exemplary levels of contaminating amplicons. For example, up to 5%, 10%, 25%, 50%, 75%, or even 100% of the amplification sites in an array can have some contaminating amplicons. It will be understood that in an array or other collection of sites, at least 50%, 75%, 80%, 85%, 90%, 95% or 99% or more of the sites can be clonal or apparently clonal.

In some embodiments, kinetic exclusion can occur when a process occurs at a sufficiently rapid rate to effectively exclude another event or process from occurring. Take for example the making of a nucleic acid array where sites of the array are randomly seeded with target nucleic acids from a solution and copies of the target nucleic acid are generated in an amplification process to fill each of the seeded sites to capacity. In accordance with the kinetic exclusion methods of the present disclosure, the seeding and amplification processes can proceed simultaneously under conditions where the amplification rate exceeds the seeding rate. As such, the relatively rapid rate at which copies are made at a site that has been seeded by a first target nucleic acid will effectively exclude a second nucleic acid from seeding the site for amplification. Kinetic exclusion amplification methods can be performed as described in detail in the disclosure of U.S. Pat. Publ. No. 2013/0338042, which is hereby incorporated by reference in its entirety.

Kinetic exclusion can exploit a relatively slow rate for initiating amplification (e.g. a slow rate of making a first copy of a target nucleic acid) vs. a relatively rapid rate for making subsequent copies of the target nucleic acid (or of the first copy of the target nucleic acid). In the example of the previous paragraph, kinetic exclusion occurs due to the relatively slow rate of target nucleic acid seeding (e.g. relatively slow diffusion or transport) vs. the relatively rapid rate at which amplification occurs to fill the site with copies of the nucleic acid seed. In another exemplary embodiment, kinetic exclusion can occur due to a delay in the formation of a first copy of a target nucleic acid that has seeded a site (e.g. delayed or slow activation) vs. the relatively rapid rate at which subsequent copies are made to fill the site. In this example, an individual site may have been seeded with several different target nucleic acids (e.g. several target nucleic acids can be present at each site prior to amplification). However, first copy formation for any given target nucleic acid can be activated randomly such that the average rate of first copy formation is relatively slow compared to the rate at which subsequent copies are generated. In this case, although an individual site may have been seeded with several different target nucleic acids, kinetic exclusion will allow only one of those target nucleic acids to be amplified. More specifically, once a first target nucleic acid has been activated for amplification, the site will rapidly fill to capacity with its copies, thereby preventing copies of a second target nucleic acid from being made at the site.

An amplification reagent can include further components that facilitate amplicon formation and in some cases increase the rate of amplicon formation. An example is a recombinase. Recombinase can facilitate amplicon formation by allowing repeated invasion/extension. More specifically, recombinase can facilitate invasion of a target nucleic acid by the polymerase and extension of a primer by the polymerase using the target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolyzable analogs thereof) in a recombinase-facilitated amplification reagent to facilitate amplification. A mixture of recombinase and single stranded binding (SSB) protein is particularly useful as SSB can further facilitate amplification. Exemplary formulations for recombinase-facilitated amplification include those sold commercially as TwistAmp kits by TwistDx (Cambridge, UK). Useful components of recombinase-facilitated amplification reagent and reaction conditions are set forth in U.S. Pat. Nos. 5,223,414 and 7,399,590, each of which is hereby incorporated by reference in its entirety.

Another example of a component that can be included in an amplification reagent to facilitate amplicon formation and in some cases to increase the rate of amplicon formation is a helicase. Helicase can facilitate amplicon formation by allowing a chain reaction of amplicon formation. The process can occur more rapidly than standard PCR since a denaturation cycle (e.g. via heating or chemical denaturation) is not required. As such, helicase-facilitated amplification can be carried out isothermally. A mixture of helicase and single stranded binding (SSB) protein is particularly useful as SSB can further facilitate amplification. Exemplary formulations for helicase-facilitated amplification include those sold commercially as IsoAmp kits from Biohelix (Beverly, Mass.). Further, examples of useful formulations that include a helicase protein are described in U.S. Pat. Nos. 7,399,590 and 7,829,284, each of which is incorporated herein by reference in its entirety.

Yet another example of a component that can be included in an amplification reagent to facilitate amplicon formation and in some cases increase the rate of amplicon formation is an origin binding protein.

### Use in Sequencing

Following attachment of adaptor-target-adaptor molecules to a surface, the sequence of the immobilized and amplified adapter-target-adapter molecules is determined. Sequencing can be carried out using any suitable sequencing technique, and methods for determining the sequence of immobilized and amplified adapter-target-adapter molecules, including strand re-synthesis, are known in the art and are described in, for instance, U.S. Pat. No. 8,053,192, WO2016/130704, U.S. Pat. No. 8,895,249, and U.S. Pat. No. 9,309,502, all of which are hereby incorporated by reference in their entirety.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid can be an automated process. Preferred embodiments include sequencing-by-synthesis (“SBS”) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using y-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

As disclosed herein, nucleotide monomers include a label moiety or dye label, attached to the nucleotide via the nucleotide's 5-prime polyphosphate. Accordingly, incorporation events can be detected based on a characteristic of the label, such as fluorescence of the label. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Images can be captured following incorporation of a labeled nucleotide into a complex of an arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. During a complexation condition, a nucleotide complementary to the next available nucleotide of a substrate-bound polynucleotide may be brought into a complex with the surface-bound polynucleotide, a primer or nascent strand complementary to the substrate-bound polynucleotide, and a polymerase. A complexation condition allows for formation of a complex but not dissociation of the dye label attached to the free nucleotide, because the kinetic conditions are unfavorable to cleavage of the 5-prime polyphosphate from the nucleotide and attaching the nucleotide to the 3-prime end of the nascent strand complementary to the surface-attached polynucleotide. Fluorescence or other signal emitted by the dye label may be captured optically during a complexation condition. Upon subsequent switching to a polymerization condition, the nucleotide's 5-prime polyphosphate and attached dye label would be cleaved from the nucleotide by the polymerase as the nucleotide is attached to the 3-prime end of the nascent strand complementary to the substrate-attached polynucleotide.

In an example, different nucleotide types can be added sequentially and an image of the array can be obtained between each addition step. In such embodiments each image will show nucleic acid features that have incorporated nucleotides of a particular type. Different features will be present or absent in the different images due the different sequence content of each feature. However, the relative position of the features will remain unchanged in the images.

In particular embodiments some or all of the nucleotide monomers can include reversible terminators. In such embodiments, reversible terminators/cleavable fluorophores can include fluorophores linked to the ribose moiety via a 3′ ester linkage (Metzker, “Emerging Technologies in DNA Sequencing,” Genome Res. 15:1767-1776 (2005), which is incorporated herein by reference in its entirety). Other approaches have separated the terminator chemistry from the cleavage of the fluorescence label (Ruparel et al., “Design and Synthesis of a 3′-O-allyl Photocleavable Fluorescent Nucleotide as a Reversible Terminator for DNA Sequencing by Synthesis,” Proc. Natl. Acad. Sci. USA 102:5932-37 (2005), which is incorporated herein by reference in its entirety). Ruparel et al. described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluorophore and effectively reverses the termination. Examples of modified nucleotides are also described in U.S. Pat. Nos. 7,427,673 and 7,057,026, the disclosures of which are incorporated herein by reference in their entireties.

Additional exemplary SBS systems and methods which can be utilized with the methods and systems described herein are described in U.S. Pat. Publ. Nos. 2007/0166705, 2006/0188901, 2006/0240439, 2006/0281109, 2012/0270305, and 2013/0260372, U.S. Pat. No. 7,057,026, WO 05/065814, U.S. Pat. Publ. No. 2005/0100900, WO 06/064199, and WO 07/010,251, the disclosures of which are incorporated herein by reference in their entireties.

Some embodiments can utilize detection of four different nucleotides using fewer than four different labels. For example, SBS can be performed utilizing methods and systems described in the incorporated materials of U.S. Pat. Publ. No. 2013/0079232, which is hereby incorporated by reference in its entirety. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pat. Publ. No. 2013/0079232, which is hereby incorporated by reference in its entirety, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system including components such as pumps, valves, reservoirs, fluidic lines and the like. A flow cell can be configured and/or used in an integrated system for detection of target nucleic acids. Exemplary flow cells are described, for example, in U.S. Pat. Publ. No. 2010/0111768 and U.S. Pat. No. 8,951,781, each of which is incorporated herein by reference in its entirety. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Pat. No. 8,951,781, which is incorporated herein by reference in its entirety.

In another aspect, the disclosure provides a kit, the kit comprising (a) a plurality of different individual nucleotides as described herein and (b) packaging materials therefor. Such a kit may include (a) individual nucleotides in accordance with those described herein, where each nucleotide may have a base that is linked to a detectable label via a cleavable linker, or a detectable label linked via an optionally cleavable linker to a blocking group of formula Z, and where the detectable label linked to each nucleotide can be distinguished upon detection from the detectable label used for other three nucleotides, and (b) packaging materials therefor. The kit may include an enzyme for incorporating the nucleotide into the complementary nucleotide chain and buffers appropriate for the action of the enzyme in addition to appropriate chemicals for removal of the blocking group and a detectable label, which may be removed in the same chemical treatment step.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

In the present disclosure, reference is made to the accompanying drawings that form a part hereof, and in which is shown by way of illustration specific embodiments which may be practiced. These embodiments are described in detail to enable those skilled in the art to practice the disclosure, and it is to be understood that other embodiments may be utilized and that structural, logical and electrical changes may be made without departing from the scope of the present disclosure. The following description of example embodiments is, therefore, not to be taken in a limited sense.

The present disclosure may be further illustrated by reference to the following examples.

### EXAMPLES

The following examples are intended to illustrate, but by no means are intended to limit, the scope of the present disclosure as set forth in the appended claims.

### Example 1—Sequencing Chemistry to Enable Scarless SBS

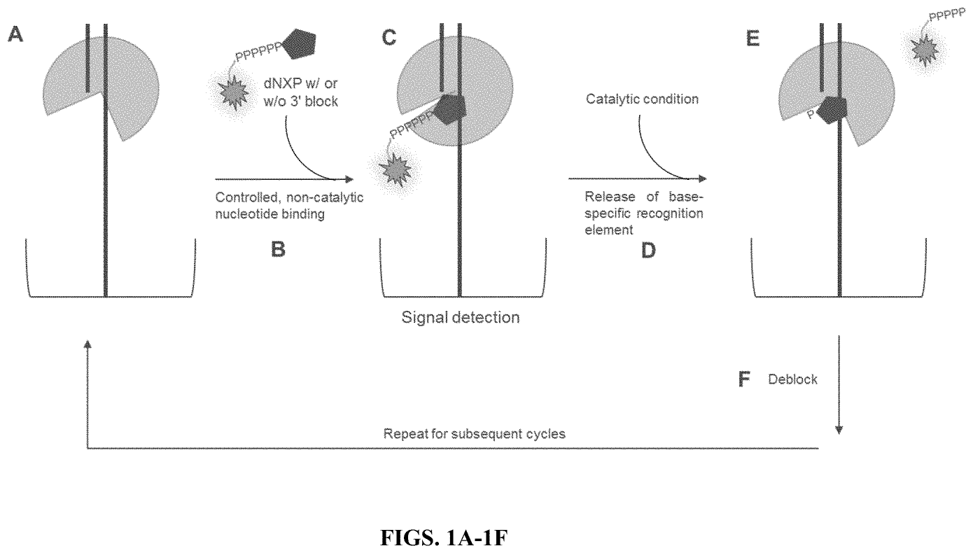

Here, a sequencing chemistry to enable scarless SBS is proposed. In this scheme, detection of the fluorescent signal occurs once the nucleotide and the polymerase are bound to the clustered DNA, opposite to the template strand, but prior to actual nucleotide incorporation (FIGS. 1A-1F). This method uses controlled catalysis in which the chemical incorporation of the nucleotide is either paused long enough or completely prevented in order to detect the signal and call the correct base.

The ability to control catalysis by pausing during the nucleotide binding step, prior to incorporation, can be also useful in single-molecule sequencing, in which the high speed of incorporation kinetics can lead to missed calls, whether through short pulse widths or short interpulse distances.

In one example, stable binding of a nucleotide substrate carrying a dye label by a polymerase-P/T complex on the surface of a flowcell occurs under non-catalytic conditions, followed by washing away of excess nucleotide in solution. Maintained non-catalytic conditions stabilize the nucleotide-polymerase-P/T ternary complex while the base is identified by its respective dye label, and, once signal detection (and thus base calling) has been achieved, the system switches from non-incorporating conditions, to incorporating conditions, by exchanging solutions. Examples of complexation (e.g., non-catalytic) conditions and polymerization (e.g., catalytic) conditions are described herein. In the presence of the catalytic condition, the DNA polymerase incorporates the nucleotide to the DNA, causing dissociation of the leaving group, which carries with it the fluorescent dye (FIGS. 1A-1F). In principle, nucleotides that, in addition to the 5′ terminal phosphate modification, contain a 3′ reversible terminator (e.g. AZM group) may be used, as currently used in traditional SBS. In this manner, precise control of nucleotide incorporation is possible to enable in each cycle the extension of a single nucleotide per DNA strand, particularly in further embodiments to be described in FIGS. 1A-1F.

A schematic of scarless SBS cycle is depicted in FIGS. 1A-1F. The polymerase is bound to primed DNA that is clustered on a flowcell surface (FIG. 1A). The nucleotide substrate carrying a 5′-phosphate label is introduced under conditions which control catalysis, pausing polymerase incorporation kinetics and retaining the label on the 5′ phosphate (FIG. 1B). Depending on the mode of detection, excess substrates may be washed away after binding. In some embodiments (particularly when the excess substrate is not washed away prior to detection) the nucleotide can carry a 3′-block to prevent multiple nucleotide incorporation events upon introduction of catalytic conditions. The signal per cluster is measured while the nucleotide substrate and its 5′-phosphate label are still bound, prior to catalysis (FIG. 1C). The conditions of the flowcell are changed such that catalysis can be promoted and the 5′ phosphate label is released from the cluster (FIG. 1D). Again, presence of a 3′-block in embodiments that do not employ washing away of excess substrate after nucleotide binding will be necessary here to enable only single extension events. The resulting DNA product contains a natural nucleotide (FIG. 1E). Some embodiments employ a nucleotide substrate with a 3′-block, in those cases a subsequent deblocking step is needed to prepare the cluster for subsequent cycles (FIG. 1F).

To enable careful control of catalysis, a number of approaches may be used. Pausing of the catalytic cycle requires non-incorporating conditions, which can created by non-catalytic metal (e.g. Ca2+, Zn2+, Co2+, Ni2+, Eu2+, Sr2+, Ba2+, Fe2+, Eu2+ and mixtures thereof), non-competitive inhibitors, competitive catalytic inhibitor, changes to nucleotide substrate to slow or prevent chemistry (non-bridging thiol or bridging nitrogen, inhibitor label), enzyme mutations to slow or prevent chemistry under certain conditions, solvent additives (ethanol, methanol, THF, dioxane, DMA, DMF, DMSO), D20 and ratios thereof, pH, and temperature.

After signal detection, incorporating conditions can be introduced that wash away non-incorporating conditions and enable release of the label. Catalytic metal including Mn2+ and/or Mg2+ will promote catalysis.

A reversible allosteric inhibitor or non-competitive polymerase inhibitor could be included. This can provide a similar benefit to the inclusion of 3′ reversible terminators by enabling stable formation of a ternary complex with control against release of the dye label from contaminating amounts of catalytic metal. Use of an allosteric/non-competitive inhibitor could “knock-out” or reduce catalysis from contaminating catalytic metal ions. The local concentration of the attached inhibitor will be quite high, so even an otherwise weak inhibitor may provide quite effective inhibition. Presumably the inhibition could be overcome using various strategies. For instance, one such inhibitor is pH-dependent, so a pH consistent with inhibition could be used with calcium for detection, then the pH could be changed to a non-inhibitory state along with the introduction of a catalytic metal like Mg2+. Specifically, the inhibition was pH dependent and could be released by Mg(II) ions in a competitive manner suggesting that electrostatic interactions are important for inhibition and that the binding sites for aminoglycosides overlap with Mg(II) ion binding sites. See Thuresson et al., “Inhibition of Poly(A) Polymerase by Aminoglycosides,” Biochimie 89:1221-27 (2007) and Ren et al., “Inhibition of Klemow DNA Polymerase and poly(A)-Specific Ribonuclease by Aminoglycosides,” RNA 8:1393-400 (2002), both of which are hereby incorporated by reference in their entirety. Kinetic analysis has revealed that aminoglycosides of the neomycin and kanamycin families behaved as mixed non-competitive inhibitors. See Thuresson et al., “Inhibition of Poly(A) Polymerase by Aminoglycosides,” Biochimie 89:1221-27 (2007) and Ren et al., “Inhibition of Klemow DNA Polymerase and poly(A)-Specific Ribonuclease by Aminoglycosides,” RNA 8:1393-400 (2002), both of which are hereby incorporated by reference in their entirety. Other potential inhibitors include pyrophosphate analogs such as and melanin.

The gamma phosphate could include an inhibitor that is not reversible, and binds to the polymerase molecule after incorporation (deactivating it), while creating a locked ternary complex. For instance, the inhibitor could bind to a cysteine near the enzyme active site after incorporation. Irreversible inhibition could also occur as a result of a non-hydrolyzable bond between the 3′-OH and the incoming nucleotide. In these cases, the label is either effectively transferred to the polymerase or prevented from being released from the incorporated nucleotide, permitting detection while creating a complex that does not dissociate. In this embodiment, harsh chemical treatment followed by polymerase-P/T complex regeneration may be required to complete a cycle and enable subsequent bases to be incorporated.

Also included in the present disclosure is the use of inhibitors (other than non-catalytic metals) that are not attached to the gamma phosphate to stabilize pre-catalytic complex formation. These could be used instead of, or in addition to, non-catalytic metals, for more complete control. For example, as discussed above, changes to pH, aminoglycosides, pyrophosphate analogs and melanin could be used.

These strategies can be extended to enable a scarless, single-molecule SBS system.

What is claimed:

1. A method comprising:
a) contacting a polymerase with a template polynucleotide and a plurality of free nucleotides, wherein the template polynucleotide is hybridized to a complementary polynucleotide comprising a 3′ end overhung by a 5′ terminal fragment of the template polynucleotide, and the plurality of free nucleotides comprise a compound of Formula (I): 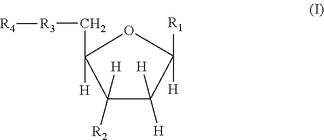
wherein R₁ comprises a nitrogenous base selected from adenine, guanine, cytosine, thymine and uracil; R₂ consists of —O—R₂ wherein R₂ is H or Z wherein Z is a removable protecting group comprising an azido group; R₃ comprises a linker comprising three or more phosphate groups; and R₄ comprises a fluorescent label;
wherein said contacting occurs under a complexation condition, the complexation condition effective to form a complex but not effective to form polymerization, wherein the complex comprises the polymerase, the template polynucleotide, the complementary polynucleotide, and one of the plurality of free nucleotides that is complementary to a first nucleotide of the 5′ terminal fragment of the template polynucleotide;
b) detecting a signal from the fluorescent label; and
c) exposing the complex to a polymerization condition.

2. The method of claim 1, wherein R₂ consists of —O—R₂ wherein R₂ is Z wherein Z is a removable protecting group comprising an azido group.

3. The method of claim 1, wherein the template polynucleotide is one of a plurality of template polynucleotides attached to a substrate.

4. The method of claim 3, wherein the plurality of template polynucleotides attached to the substrate comprise a cluster of copies of a library polynucleotide.

5. The method of claim 1, further comprising:
repeating steps a) through c) one or more times.

6. The method of claim 1, wherein the polymerization condition comprises a concentration of Mg²⁺ ions, wherein the concentration of Mg²⁺ ions is in a range of about 0.1 mM to about 10 mM, or a concentration of Mn²⁺ ions, wherein the concentration of Mn²⁺ ions is in a range of about 0.1 mM to about 10 mM.

7. The method of claim 1, wherein the complexation condition comprises a non-catalytic metal cation.

8. The method of claim 7, wherein the non-catalytic metal cation is selected from the group consisting of one or more of Ca²⁺, Zn²⁺, Co²⁺, Ni²⁺, Eu²⁺, Sr²⁺, Ba²⁺, Fe²⁺, and Eu²⁺.

9. The method of claim 7, wherein the concentration of the non-catalytic metal cation is less than or equal to about 10 mM.

10. The method of claim 1, wherein the complexation condition comprises a chelating agent.

11. The method of claim 10, wherein the chelating agent is selected from the group consisting of ethylene glycol-bis(β-aminoethyl ether)-N,N,N′,N′-tetraacetic acid (EGTA), nitriloacetic acid, tetrasodium iminodisuccinate, ethylene glycol tetraacetic acid, polyaspartic acid, ethylenediamine-N,N′-disuccinic acid (EDDS), methylglycindiacetic acid (MGDA), and a combination thereof.

12. The method of claim 10, wherein the complexation condition further comprises an inhibitor selected from the group consisting of a non-competitive inhibitor, a competitive inhibitor, and a combination thereof.

13. The method of claim 1, wherein the complexation condition comprises a pH that is less than about 6.

14. The method of claim 1, wherein the polymerization condition comprises a pH that is greater than or equal to about 6.

15. The method of claim 1, wherein the complexation condition comprises a non-competitive inhibitor.

16. The method of claim 15, wherein the non-competitive inhibitor is selected from the group consisting of an aminoglycoside, a pyrophosphate analog, a melanin, a phosphonoacetate, a hypophosphate, a rifamycin, and a combination thereof.

17. The method of claim 1, wherein the complexation condition comprises a competitive inhibitor.

18. The method of claim 17, wherein the competitive inhibitor is selected from the group consisting of aphidicolin, beta-D-arabinofuranosyl-CTP, amiloride, dehydroaltenusin, and a combination thereof.

19. The method of claim 1, wherein the complexation condition comprises a solvent additive.

20. The method of claim 19, wherein the solvent additive is selected from the group consisting of ethanol, methanol, tetrahydrofuran, dioxane, dimethylamine, dimethylformamide, dimethyl sulfoxide, lithium, L-cysteine, and a combination thereof.

21. The method of claim 1, wherein the complexation condition comprises deuterium.

22. The method of claim 2, wherein the 3′-hydroxy blocking group comprises a reversible terminator.

23. The method of claim 22, wherein the reversible terminator comprises an azidomethyl group or an acetal group.

24. The method of claim 22, further comprising:
removing the reversible terminator after the 3′ end of the complementary polynucleotide is covalently bonded to a phosphate group of the linker.

25. The method of claim 1, wherein the free nucleotide further comprises a non-bridging thiol or a bridging nitrogen.

26. The method of claim 1, wherein the polymerase comprises a mutation.

27. The method of claim 26, wherein the mutation modifies speed of one or more of steps a) through c).
